(19) United States
(12) Patent Application Publication
(10) Pub. No.: US 2025/0057924 A1
(43) Pub. Date: Feb. 20, 2025

(54) METHODS AND COMPOSITIONS FOR IMPROVED MOLECULAR THERAPIES OF MULTIGENIC DISEASES

(71) Applicant: Robert E. Sobol, San Diego, CA (US)

(72) Inventor: Robert E. Sobol, San Diego, CA (US)

(21) Appl. No.: 18/722,528

(22) PCT Filed: Dec. 19, 2022
(86) PCT No.: PCT/US2022/053404

§ 371 Date: Jun. 20, 2024

Related U.S. Application Data
(60) Provisional application No. 63/291,722, filed on Dec. 20, 2021.

(51) Int. Cl. A61K 38/46; A61K 31/7088; A61K 38/17; A61K 38/19; A61K 38/20; A61K 38/21; A61K 38/30; A61K 47/68; A61K 48/00; C12N 9/22; C12N 15/11

(52) U.S. Cl. CPC A61K 38/465; A61K 31/7088; A61K 38/1709; A61K 38/177; A61K 38/19; A61K 38/195; A61K 38/20; A61K 38/21; A61K 38/30; A61K 47/6807; A61K 47/6849; A61K 47/6851; A61K 48/0033; C12N 9/22; C12N 15/11; C12N 2310/20

(57) ABSTRACT

Provided herein are methods and compositions that enhance the efficacy and safety of gene editing for treating and preventing multigenic diseases.

### CROSS-REFERENCE

This application claims the benefit of U.S. Provisional Application No. 63/291,722, filed Dec. 20, 2021, which application is incorporated herein by reference its entirety.

### FIELD

The present disclosure relates generally to the fields of biology, medicine, multigenic diseases such as cancer, hyperproliferative disorders and gene editing. More particularly, it concerns methods and compositions that enhance the efficacy of genetic therapies for cancer and other multigenic diseases.

### BACKGROUND

While significant progress has been made in defining the genetic abnormalities that contribute to the pathogenesis of many diseases, the ability to address these deficiencies through genetic therapies remains limited particularly for multigenic diseases. For example, knowledge of the genetic aberrancies that underlie the etiology and progression of cancer have continuously increased over the past 20 years. In this regard, the Cancer Genome Atlas (TCGA) project, begun in 2006, has molecularly characterized over 20,000 primary cancer and matched normal samples spanning 33 cancer types. The TCGA has generated over 2.5 petabytes of genomic, epigenomic, transcriptomic, and proteomic data. The data is publicly available for anyone in the research community to use.

Concurrently, there has been substantial progress made in gene therapy and gene editing technologies to administer therapeutic genes and modify the genome. Genome editing approaches involve the use of meganucleases, zinc finger nucleases (ZFNs), transcription activator-like effector-based nucleases (TALEN), and the clustered regularly interspaced short palindromic repeats (CRISPR/Cas) systems. Precision genome editing has been developed through various approaches, including oligonucleotide-directed mutagenesis (ODM), base editing, prime editing, homology directed repair (HDR), microhomology-mediated end joining (MMEJ), non-homology end joining (NHEJ) and alternative non-homologous end joining (Alt-NHEJ) pathways.

However, major difficulties remain in treating multigenic diseases, such as cancer, due to the multitude of complementary, amplifying abnormalities that are present which defy amelioration by a single therapy or genetic modification. In this regard, cancers are typically treated with multiple therapies which aim to engage several mechanisms of action. While these multiple therapies have resoundingly proven more efficacious than single treatments, they remain significantly limited by the varying bioavailabilities of their component agents to simultaneously reach target cells and by their cumulative toxicities in normal tissues. Furthermore, many of the molecular targets deemed important for potentially efficacious cancer treatment are either undruggable by conventional methods or trigger resistance mechanisms. Hence, while many potentially powerful therapies can be envisioned based upon cancer's molecular pathogenesis, their practical combination is limited by toxicities in normal tissues and in many cases, key pathways are either undruggable by conventional methods or result in resistance development. Furthermore, current gene editing technologies are challenged to make in vivo changes to a single disease-related gene demonstrating very low efficiencies (typically 5% or less) for in vivo gene corrections or gene knock ins which are also limited to a single gene locus. Current in vivo gene editing techniques are therefore limited in their ability to effectively address multigenic diseases, particularly those disorders with many genetic aberrancies such as cancer.

The limitations and inadequacies of current cancer therapies are further exemplified by the fact that despite the identification of numerous molecular targets and the development of a variety of genetically targeted treatments and technologies, cancer remains a leading cause of morbidity and mortality worldwide. According to estimates from the International Agency for Research on Cancer (IARC), in 2018 there were 17.0 million new cancer cases and 9.5 million cancer deaths worldwide. By 2040, the global burden is expected to grow to 27.5 million new cancer cases and 16.3 million cancer deaths.

### SUMMARY

There is an unmet need for improved genetically targeted therapies particularly for those diseases that have multigenic etiologies and conventionally undruggable components. This disclosure meets this unmet need by providing compositions and methods that overcome the deficiencies of current approaches by providing an efficacious and well tolerated in vivo gene editing therapy that concurrently inhibits multiple pathogenic targets in a single treatment while concurrently expressing therapeutic agents. Importantly, the approach can also effectively address any molecular target including those undruggable by conventional methods. Additionally, and significantly, the disclosed compositions and methods permit the realization of targeted therapy combinations that are too toxic for practical use by conventional treatments and which can reverse treatment resistance. Overall, the disclosed treatments provide genomic editing compositions and methods that work together to provide an unprecedented ability to address multiple molecular abnormalities not previously achievable in the treatment of multigenic diseases. The approach is descriptively named “synergic editing”.

In one aspect, a vector is provided comprising: (a) at least one donor therapeutic mRNA comprising a nucleic acid sequence encoding at least one therapeutic moiety; (b) a genome editing endonuclease or a nucleic acid sequence encoding a genome editing endonuclease; and (c) one or more guide RNA (gRNA) sequences for guiding the genome editing endonuclease to cleave at least one pathogenic gene of interest, thereby resulting in the disruption of the pathogenic gene of interest and expression of the at least one therapeutic moiety. In some cases, the vector further comprises a targeting moiety. In some cases, the targeting moiety is a cancer cell targeting moiety. In some cases, the targeting moiety is selected from the group consisting of: an antibody, an antibody fragment, a nanobody, a receptor ligand, and any combination thereof. In some cases, the targeting moiety is configured to bind to a target and deliver the vector to cancer cells, pre-cancerous cells, or pathogenic cells in a tumor microenvironment. In some cases, the targeting moiety binds to a target selected from the group consisting of: EGFR, HER2, CD19, CD20, CD22, CD33, CD38, BCMA, Nectin-4, Trop-2, tissue factor, GD2, any target listed in Table 5, and any combination thereof. In some cases, the nucleic acid sequence encoding the at least one therapeutic moiety is selected from the group consisting of: a tumor suppressor gene, a pro-apoptotic gene, an immune stimulatory gene, a suicide gene, an anti-cancer gene silenced by hypermethylation or other epigenetic mechanisms, a secreted decoy receptor gene, and any combination thereof. In some cases, the at least one therapeutic moiety is selected from the group consisting of: p53, PTEN, Rb, IL24, APC, BAX, BAK, BCLX, an interleukin, an interferon, a CD122/132 agonist, a chemokine, HSP 70/90, Calreticulin, HMGB1, a Toll Like Receptor, CGAS, STING1, ARHI, RASSF1A, DLEC1, SPARC, DAB2, PLAGL1, RPS6KA2, PTEN, OPCML, BRCA2, ARL11, WWOX, TP53, DPH1, BRCA1, mTORC2, PEG3, IGF2, SAT2, p16INK4A, p14ARF, IRF3/7, any therapeutic moiety listed in Table 3, and any combination thereof. In some cases, the at least one therapeutic moiety is a prodrug modifying enzyme. In some cases, the pathogenic gene of interest is selected from the group consisting of: an oncogene, an angiogenic gene, an immune suppressive gene, an anti-apoptotic gene, a therapy resistance gene, and any combination thereof. In some cases, the pathogenic gene of interest is selected from the group consisting of: HDM2/HDM4/HDMX, BCL2, BCL-XL, BIRC5/Survivin, AKT, PLK1, E2F1, CMYC, NFKappaB, CCND1-3, KRAS, FOS, JUN, JAK, STAT, PKA, CREB, TGFB, Beta-Catenin, IL10, PD1, PDL1, MET, AXL, SRC, RAS, PIK3, PGP2, GST, MRP1, BCRP/ABCG2, any gene listed in Table 1, and any combination thereof. In some cases, the genome editing endonuclease is a CRISPR-associated endonuclease. In some cases, the CRISPR-associated endonuclease is selected from the group consisting of: Cas9, Cas12a, Cas12b, Cas12e, Cas13a, Cas13b, Cas14, Cas-theta, CasX, CasY, any CRISPR-associated endonuclease listed in Table 2, and any combination thereof. In some cases, the one or more gRNA sequences is selected from any gRNA nucleic acid sequence described in Table 1. In some cases, the genome editing endonuclease is Cas9. In some cases, the vector is a lipid nanoparticle. In some cases, the vector is a viral vector. In some cases, the viral vector is selected from the group consisting of: an adenovirus, an adeno-associated virus, a lentivirus, a herpes virus, and any derivative thereof.

In another aspect, a method of treating a multigenic disease in a subject is provided, the method comprising administering an effective amount of a vector of any one of the preceding to the subject, thereby treating the multigenic disease. In some cases, the multigenic disease is cancer. In some cases, the method further comprises administering to the subject at least one additional anti-cancer therapy. In some cases, the at least one additional anti-cancer treatment is selected from the group consisting of: surgical therapy, chemotherapy, radiation therapy, hormonal therapy, immunotherapy, small molecule therapy, receptor kinase inhibitor therapy, anti-angiogenic therapy, cytokine therapy, cryotherapy, radioablation, a biological therapy, a monoclonal antibody, siRNA, miRNA, an antisense oligonucleotide, a ribozyme, a gene editor, cellular therapy, gene therapy, and any combination thereof. In some cases, the cancer is selected from the group consisting of: melanoma, non-small cell lung, small-cell lung, lung, hepatocarcinoma, retinoblastoma, astrocytoma, glioblastoma, leukemia, neuroblastoma, head, neck, breast, pancreatic, prostate, renal, bone, testicular, ovarian, mesothelioma, cervical, gastrointestinal, urogenital, respiratory tract, hematopoietic, musculoskeletal, neuroendocrine, carcinoma, sarcoma, central nervous system, peripheral nervous system, lymphoma, brain, colon cancer, bladder cancer, and any combination thereof. In some cases, the subject is a human. In some cases, the administering is selected from the group consisting of intravenous, intraarterial, intravascular, intrapleural, intraperitoneal, intratracheal, intratumoral, intrathecal, intramuscular, endoscopic, intralesional, percutaneous, subcutaneous, region, stereotactical, direct injection, and perfusion administration. In some cases, the administering comprises administering the vector to the subject more than once.

In another embodiment, the present disclosure provides a single vector composition effective in the treatment of multigenic diseases that provides synergic disruption of pathogenic genes with concurrent expression of a beneficial therapeutic gene sequence. The net effect of the composition in multigenic diseases is providing synergic expression of therapeutic nucleic acids while concurrently decreasing the expression of pathogenic genes in the same treatment.

According to an aspect of some embodiments of the present disclosure there is provided a vector comprising a donor gene expression nucleic acid sequence containing at least one gene editing endonuclease cleavage site; and a genome editing endonuclease protein or mRNA; and one or more nucleic acid sequences guiding said genome editing endonuclease to cleave the donor gene editing insertion/expression sequence and at least one pathogenic gene of interest resulting in the disruption of the pathogenic gene and insertion/expression of the donor gene editing insertion/expression nucleic acid sequence.

According to an aspect of some embodiments of the present disclosure there is provided the method of preparing a vector comprising a donor gene editing insertion/expression nucleic acid sequence containing at least one gene editing endonuclease cleavage site; and a genome editing endonuclease protein or mRNA; and one or more nucleic acid sequences guiding said genome editing endonuclease to cleave the donor gene editing insertion/expression sequence and at least one pathogenic gene of interest resulting in the disruption of the pathogenic gene and insertion/expression of the donor gene editing insertion/expression nucleic acid sequence.

According to an aspect of some embodiments of the present disclosure there is provided the vector comprising a therapeutic mRNA nucleic acid expression sequence and a genome editing endonuclease protein or mRNA; and one or more nucleic acid sequences guiding said genome editing endonuclease to cleave at least one pathogenic gene of interest resulting in the disruption of the pathogenic gene and expression of the therapeutic mRNA nucleic acid sequence.

According to an aspect of some embodiments of the present disclosure there is provided the method of preparing a vector comprising a therapeutic mRNA nucleic acid expression sequence and a genome editing endonuclease protein or mRNA; and one or more nucleic acid sequences guiding said genome editing endonuclease to cleave at least one pathogenic gene of interest resulting in the disruption of the pathogenic gene and expression of the therapeutic mRNA nucleic acid sequence.

According to an aspect of some embodiments of the present disclosure there is provided the vectors comprising at least one donor therapeutic DNA template containing at least one syn-gRNA or Usyn-gRNA guide RNA endonuclease cleavage site; or at least one donor therapeutic mRNA; and a genome editing endonuclease protein or mRNA; and one or more syn-gRNA/single guide RNA or Usyn-gRNA nucleic acid sequences guiding said genome editing endonuclease to cleave the donor therapeutic DNA template and at least one pathogenic gene of interest resulting in the disruption of the pathogenic gene and expression of the donor therapeutic nucleic acid sequence.

According to an aspect of some embodiments of the present disclosure there is provided the methods of preparing a vector comprising at least one donor therapeutic DNA template containing at least one syn-gRNA or Usyn-gRNA guide RNA endonuclease cleavage site; or at least one donor therapeutic mRNA; and a genome editing endonuclease protein or mRNA; and one or more syn-gRNA/single guide RNA or Usyn-gRNA nucleic acid sequences guiding said genome editing endonuclease to cleave the donor therapeutic DNA template and at least one pathogenic gene of interest resulting in the disruption of the pathogenic gene and expression of the donor therapeutic nucleic acid sequence.

According to an aspect of some embodiments of the present disclosure there is provided the vectors further comprising a targeting moiety.

According to an aspect of some embodiments of the present disclosure there is provided the vector or the method to treat a multigenic disease.

According to an aspect of some embodiments of the present disclosure there is provided the methods to treat a monogenic disease where a beneficial effect is obtained from concurrent gene disruption and gene expression by synergic editing.

According to an aspect of some embodiments of the present disclosure there is provided the methods wherein the subject is further administered at least one additional therapy.

According to an aspect of some embodiments of the present disclosure there is provided the vector or the method where the multigenic disease to be treated is cancer.

According to an aspect of some embodiments of the present disclosure there is provided the vectors or the method of wherein the donor therapeutic DNA nucleic acid expression sequence or donor therapeutic mRNA nucleic acid expression sequence encodes a tumor suppressor gene, a pro-apoptotic gene, an immune stimulatory gene, a suicide gene, an anti-cancer gene silenced by hypermethylation or other epigenetic mechanisms, or a secreted decoy receptor gene.

According to an aspect of some embodiments of the present disclosure there is provided the vectors or the method wherein the pathogenic gene of interest for disruption is an oncogene, an angiogenic gene, an immune suppressive gene, an anti-apoptotic gene, or a therapy resistance gene.

According to an aspect of some embodiments of the present disclosure there is provided the vectors or the methods wherein the genome editing endonuclease is a CRISPR associated gene editing endonuclease protein or mRNA.

According to an aspect of some embodiments of the present disclosure there is provided the vectors or the methods wherein said CRISPR-associated endonuclease is Cas9, Cas12a, Cas12b, Cas12e, Cas13a, Cas13b, Cas14, Cas-theta, CasX or CasY or listed in Table 2.

According to an aspect of some embodiments of the present disclosure there is provided the vectors or the methods wherein said nucleic acid sequence guiding said genome editing endonuclease is a CRISPR system guide RNA, syn-gRNA, single guide RNA, or Usyn-gRNA.

According to an aspect of some embodiments of the present disclosure there is provided the vectors or methods wherein the donor therapeutic DNA or donor therapeutic mRNA expression sequence encodes p53, PTEN, Rb, IL24, APC, BAX, BAK, BCLX, an interleukin, an interferon, a CD122/132 agonist, a chemokine, HSP 70/90, Calreticulin, HMGB1, a Toll Like Receptor, CGAS, STING1, ARHI, RASSF1A, DLEC1, SPARC, DAB2, PLAGL1, RPS6KA2, PTEN, OPCML, BRCA2, ARL11, WWOX, TP53, DPH1, BRCA1, mTORC2, PEG3, IGF2, SAT2, p16INK4A, p14ARF, IRF3/7 or a gene listed in Table 3.

According to an aspect of some embodiments of the present disclosure there is provided the vectors or method wherein the donor therapeutic DNA or donor therapeutic mRNA expression sequence encodes a prodrug modifying enzyme selected from Table 4 administered with its paired prodrug.

According to an aspect of some embodiments of the present disclosure there is provided the vectors or methods wherein the disrupted pathogenic gene of interest is HDM2/HDM4/HDMX, BCL2, BCL-XL, BIRC5/Survivin, AKT, PLK1, E2F1, CMYC, NFKappaB, CCND1-3, KRAS, FOS, JUN, JAK, STAT, PKA, CREB, TGFB, Beta-Catenin, IL10, PD1, PDL1, MET, AXL, SRC, RAS, PIK3, PGP2, GST, MRP1, BCRP/ABCG2 or a gene listed in Table 1.

According to an aspect of some embodiments of the present disclosure there is provided the vectors or methods wherein the at least one nucleic acid sequence guiding said genome editing endonuclease to the pathogenic gene of interest for disruption comprises the syn-gRNAs/single guide RNAs with Cas9 listed in Table 1.

According to an aspect of some embodiments of the present disclosure there is provided the vector or methods wherein the donor therapeutic DNA with universal synergic cleavage/insertion sequences at the 5′ or 5′ and 3′ positions for use with Usyn-gRNA are GGAATCCTCGCGTGCGAAGTngg (SEQ ID NO: 79) or GAGCTGGACGGCGACGTAAAngg (SEQ ID NO: 80).

According to an aspect of some embodiments of the present disclosure there is provided the vectors or methods wherein the targeting moiety is an antibody, antibody fragment, nanobody, or receptor ligand.

According to an aspect of some embodiments of the present disclosure there is provided the vectors or methods wherein the targeting moiety delivers the vector to cancer cells, pre-cancerous cells or pathogenic cells in the tumor microenvironment.

According to an aspect of some embodiments of the present disclosure there is provided the vectors or methods where the targeting moiety binds to EGFR, HER2, CD19, CD20, CD22, CD33, CD38, BCMA, Nectin-4, Trop-2, tissue factor, GD2 or a target listed in Table 5.

According to an aspect of some embodiments of the present disclosure there is provided a lipid nanoparticle delivering the vectors.

According to an aspect of some embodiments of the present disclosure there is provided a viral particle delivering the vectors.

According to an aspect of some embodiments of the present disclosure there is provided a method of treating cancer in a subject comprising administering an effective amount of the vectors to the subject.

According to an aspect of some embodiments of the present disclosure there is provided the methods wherein the subject is further administered at least one additional anti-cancer therapy.

According to an aspect of some embodiments of the present disclosure there is provided the methods wherein the at least one additional anticancer treatment is surgical therapy, chemotherapy, radiation therapy, hormonal therapy, immunotherapy, small molecule therapy, receptor kinase inhibitor therapy, anti-angiogenic therapy, cytokine therapy, cryotherapy, radioablation or a biological therapy. In some aspects, the biological therapy is a monoclonal antibody, siRNA, miRNA, antisense oligonucleotide, ribozyme, gene editing, cellular therapy or gene therapy.

According to an aspect of some embodiments of the present disclosure there is provided the method of treatment wherein the subject is a human.

The method of claims 22-25, wherein the cancer is melanoma, non-small cell lung, small-cell lung, lung, hepatocarcinoma, retinoblastoma, astrocytoma, glioblastoma, leukemia, neuroblastoma, head, neck, breast, pancreatic, prostate, renal, bone, testicular, ovarian, mesothelioma, cervical, gastrointestinal, urogenital, respiratory tract, hematopoietic, musculoskeletal, neuroendocrine, carcinoma, sarcoma, central nervous system, peripheral nervous system, lymphoma, brain, colon or bladder cancer.

According to an aspect of some embodiments of the present disclosure there are provided vectors or methods of treating cancer or another disease in a subject comprising administering an effective amount of vectors to the subject intravenously, intraarterially, intravascularly, intrapleurally, intraperitoneally, intratracheally, intratumorally, intrathecally, intramuscularly, endoscopically, intralesionally, percutaneously, subcutaneously, regionally, stereotactically, or by direct injection or perfusion. In certain aspects, the subject is administered the vector more than once.

In some aspects, the additional immunotherapy comprises a cytokine, such as GM-CSF, an interleukin (e.g., IL-2) and/or an interferon (e.g., IFNa) or heat shock proteins. In certain aspects, the immunotherapy comprises a co-stimulatory receptor agonist, a stimulator of innate immune cells, or an activator of innate immunity. In certain aspects, the co-stimulatory receptor agonist is an anti-OX40 antibody, anti-GITR antibody, anti-CD 137 antibody, anti-CD40 antibody, or an anti-CD27 antibody. In some aspects, the stimulator of immune cells is an inhibitor of a cytotoxicity-inhibiting receptor or an agonist of immune stimulating toll like receptors (TLR). In some aspects, the cytotoxicity-inhibiting receptor is an inhibitor of NKG2A/CD94 or CD96 TACTILE. In some aspects, the TLR agonist is a TLR7 agonist, TLR8 agonist, or TLR9 agonist. In some aspects, the immunotherapy comprises a combination of a PD-L1 inhibitor, a 4-1BB agonist, and an OX40 agonist. In certain aspects, the immunotherapy comprises a stimulator of interferon genes (STING) agonist. In some aspects, the activator of innate immunity is an IDO inhibitor, TGF inhibitor, or IL-10 inhibitor. In some aspects, when these immunotherapies are proteins, they may be delivered as polypeptides or their corresponding nucleic acids administered by replication competent and/or replication incompetent viral and/or non-viral gene therapy. In some aspects, the chemotherapy comprises a DNA damaging agent, such as gamma-irradiation, X-rays, UV-irradiation, microwaves, electronic emissions, adriamycin, 5-fluorouracil (5FU), capecitabine, etoposide (VP-16), camptothecin, actinomycin-D, mitomycin C, cisplatin (CDDP), or hydrogen peroxide.

In certain aspects, the additional immune therapy is a CD122/CD132 agonist that preferentially binds to the CD122/CD132 receptor complex and has lower affinity binding for CD25 or the IL15 alpha receptor as compared to the affinity binding to the CD122/CD132 receptor complex. In specific aspects, the one or more CD122/CD132 agonists are an IL-2/anti-IL-2 immune complex, an IL-15/anti-IL-15 immune complex, an IL-15/IL-15 Receptor a-IgG1-Fc (IL-15/IL-15Ra-IgG1-Fc) immune complex, PEGylated IL-2, PEGylated IL-15, IL-2 mutein and/or IL-15 mutein. The CD122/CD132 agonist may be an IL-15 mutant (e.g., IL-15N72D) bound to an IL-15 receptor a/IgG1 Fc fusion protein, such as ALT-803. In certain aspects, IL-15 is pre-complexed with IL-15Ra to preferentially bind to CD122/CD132. In particular aspects, the CD122/CD132 agonist is F42K.

In some aspects, the chemotherapy comprises a DNA damaging agent. In some embodiments, the DNA damaging agent is gamma-irradiation, X-rays, UV-irradiation, microwaves, electronic emissions, adriamycin, 5-fluorouracil (5FU), capecitabine, etoposide (VP-16), camptothecin, actinomycin-D, mitomycin C, cisplatin (CDDP), or hydrogen peroxide. In particular aspects, the DNA damaging agent is 5FU or capecitabine. In some aspects, the chemotherapy comprises a cisplatin (CDDP), carboplatin, procarbazine, mechlorethamine, cyclophosphamide, camptothecin, ifosfamide, melphalan, chlorambucil, bisulfan, nitrosurea, dactinomycin, daunorubicin, doxombicin, bleomycin, plicomycin, mitomycin, etoposide (VP16), tamoxifen, taxotere, taxol, transplatinum, 5-fluorouracil, vincristin, vinblastin, methotrexate, an HDAC inhibitor or any analog or derivative variant thereof.

In some aspects, the at least one additional cancer treatment is a protein kinase inhibitor or a monoclonal antibody that inhibits receptors involved in protein kinase or growth factor signaling pathways. For example, the protein kinase or receptor inhibitor can be an EGFR, VEGFR, AKT, Erb1, Erb2, ErbB, Syk, Bcr-Abl, JAK, Src, GSK-3, PI3K, Ras, Raf, MAPK, MAPKK, mTOR, c-Kit, eph receptor or BRAF inhibitor. In particular aspects, the protein kinase inhibitor is a PI3K inhibitor. In some embodiments, the PI3K inhibitor is a PI3K delta inhibitor. For example, the protein kinase or receptor inhibitor can be Afatinib, Axitinib, Bevacizumab, Bosutinib, Cetuximab, Crizotinib, Dasatinib, Erlotinib, Fostamatinib, Gefitinib, Imatinib, Lapatinib, Lenvatinib, Mubritinib, Nilotinib, Panitumumab, Pazopanib, Pegaptanib, Ranibizumab, Ruxolitinib, Saracatinib, Sorafenib, Sunitinib, Trastuzumab, Vandetanib, AP23451, Vemurafenib, CAL101, PX-866, LY294002, rapamycin, temsirolimus, everolimus, ridaforolimus, Alvocidib, Genistein, Selumetinib, AZD-6244, Vatalanib, P1446A-05, AG-024322, ZD1839, P276-00, GW572016, or a mixture thereof. In certain aspects, the protein kinase inhibitor is an AKT inhibitor (e.g., MK-2206, GSK690693, A-443654, VQD-002, Miltefosine or Perifosine). In certain aspects, EGFR-targeted therapies for use in accordance with the embodiments include, but are not limited to, inhibitors of EGFR/ErbB1/HER, ErbB2/Neu/HER2, ErbB3/HER3, and/or ErbB4/HER4. A wide range of such inhibitors are known and include, without limitation, tyrosine kinase inhibitors active against the receptor(s) and EGFR-binding antibodies or aptamers. For instance, the EGFR inhibitor can be gefitinib, erlotinib, cetuximab, matuzumab, panitumumab, AEE788; CI-1033, HKI-272, HKI-357, or EKB-569. The protein kinase inhibitor may be a BRAF inhibitor such as dabrafenib, or a MEK inhibitor such as trametinib.

Other objects, features and advantages of the present disclosure will become apparent from the following detailed description. It should be understood, however, that the detailed description and the specific examples, while indicating preferred embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1. Synergic Editing for Multigenic Disease with Donor Therapeutic DNA depicts synergic editing to treat multigenic diseases with donor therapeutic DNA vectors that provide synergic disruption of pathological genes with expression of therapeutic DNA.

FIG. 2. Components of Synergic Editing Donor Therapeutic DNA Vectors depicts the vector compositions and general method for synergic editing to treat multigenic diseases with donor therapeutic DNA vectors that synergically disrupt pathological genes of interest and express a therapeutic cDNA.

FIG. 3. Synergic Editing for Cancer Treatment with Donor Therapeutic DNA depicts the general vector compositions and methods for synergic editing to treat multigenic cancers with Donor Therapeutic DNA Vectors. Without the intention of any limitation, this figure provides depiction of representative Donor Therapeutic DNA Vectors for multigenic cancer treatment comprising a cancer cell targeting lipid nanoparticle (LNP) containing a tumor targeting moiety (exemplified by, but not limited to, anti-epidermal growth factor receptor antibody), donor therapeutic DNA (exemplified by, but not limited to, tumor suppressor, pro-apoptotic, immune stimulatory DNAs); synergic guide RNAs (syn-gRNAs)/single guide RNAs (sgRNAs) and endonuclease (as described in FIG. 2) that disrupt pathological cancer genes of interest (exemplified by, but not limited to, oncogenes, anti-apoptotic and immune suppressive genes) and concurrently express a therapeutic cDNA. A “universal” synergic cleavage/insertion sequence may be used in the Donor Therapeutic DNA template necessitating the addition of universal synergic guide RNA (Usyn-gRNA) to the vector. When utilized, the Usyn-gRNA/Cas complex cleaves the Donor Therapeutic DNA Template facilitating its integration into the pathogenic cancer genomic sites which are cleaved by the syn-gRNA/Cas endonuclease complexes.

FIG. 4. Synergic Editing for Multigenic Disease with Donor Therapeutic mRNA depicts the vector compositions and general method for synergic editing to treat multigenic diseases with therapeutic mRNA vectors that concurrently disrupt pathological genes of interest and express a therapeutic mRNA.

FIG. 5. Synergic Editing for Cancer Treatment with Donor Therapeutic mRNA depicts the general vector compositions and methods for synergic editing to treat multigenic cancers with Donor Therapeutic mRNA Vectors. Without the intention of any limitation, this figure provides depiction of representative Donor Therapeutic mRNA Vectors for multigenic cancer treatment comprising a cancer cell targeting lipid nanoparticle (LNP) containing a tumor targeting moiety (exemplified by, but not limited to, anti-CD38 antibody), therapeutic mRNA (exemplified by but not limited to tumor suppressor, pro-apoptotic, immune stimulatory mRNAs); synergic guide RNA and endonuclease (as described in FIG. 4) that disrupt pathological cancer genes of interest (exemplified by, but not limited to, oncogenes, anti-apoptotic and immune suppressive genes) and synergically express a therapeutic mRNA.

FIG. 6. Synergic Editing for Cancer Treatment with Donor Therapeutic Tumor Suppressor mRNA and Oncogene Knock Out depicts the general vector compositions and methods for synergic editing to treat cancers with Donor Therapeutic Tumor Suppressor mRNA and Oncogene Knockout Vectors. Without the intention of any limitation, this figure provides depiction of representative Donor Therapeutic Tumor Suppressor mRNA Vectors comprising a cancer cell targeting lipid nanoparticle (LNP) containing a tumor targeting moiety (exemplified by, but not limited to, an antibody), therapeutic tumor suppressor mRNA (exemplified by, but not limited to, TP53 mRNA); synergic/single guide RNA and endonuclease (exemplified by, but not limited to, a single guide RNA to the oncogene MDM2 and Cas9 endonuclease mRNA) that disrupt pathological cancer oncogene of interest (exemplified by, but not limited to, MDM2) and synergically express a therapeutic mRNA (exemplified by, but not limited to, TP53 mRNA).

FIG. 7A and FIG. 7B. Results of Synergic Editing for Cancer Treatment with Donor Therapeutic TP53 Tumor Suppressor mRNA and MDM2 Oncogene Knock Out depicts the results of synergic editing to treat cancers with Donor Therapeutic Tumor Suppressor mRNA and Oncogene Knockout Vectors. Without the intention of any limitation, this figure provides depiction of representative results of Synergic Editing with Donor Therapeutic Tumor Suppressor mRNA comprising a lipid nanoparticle (LNP) containing a therapeutic tumor suppressor mRNA (exemplified by, but not limited to, TP53 mRNA); synergic/single guide RNA and endonuclease (exemplified by, but not limited to, a single guide RNA to the oncogene MDM2 and Cas9 endonuclease mRNA) that disrupts pathological cancer oncogene of interest (exemplified by, but not limited to, MDM2) and synergically express a therapeutic mRNA (exemplified by, but not limited to, TP53 mRNA). The unexpected results are shown in FIG. 7A and FIG. 7B. In HepG2 hepatocellular carcinoma, treatment with either TP53 mRNA or MDM2 KO LNPs alone had no therapeutic effects with basically unchanged viability compared to the untreated control. Surprisingly, the TP53 mRNA+MDM2 KO LNP demonstrated unexpected synergic effects resulting in a greater than 40% decrease in viability which was statistically significant overall by ANOVA P=0.007. In addition, there was a statistically significant decrease in viability for the “synergic” TP53 mRNA+MDM2 KO LNP compared to either the TP53 mRNA LNP alone (P=0.0013) or the MDM2 KO LNP alone (P=0.0006). Similar unexpected and corroborating results are observed for the treatment of ovarian carcinoma. As shown in FIG. 7B for OVCAR8 ovarian carcinoma, the greatest decrease in viability (greater than 70%) was observed for the TP53 mRNA+MDM2 KO LNP group which demonstrated more than additive effects compared to the other groups with statistically confirmed synergy by ANOVA P<0.0001. Consistent with the results for hepatocellular carcinoma above, the ovarian carcinoma treatment also demonstrated a statistically significant decrease in viability for the “synergic” TP53 mRNA+MDM2 KO LNP compared to either the TP53 mRNA LNP alone (P=0.0205) or the MDM2 KO LNP alone (P=0.0001). Overall, these treatment findings demonstrate the unexpected, substantial and statistically significant synergy of the TP53 mRNA+MDM2 KO LNP compared to either the TP53 mRNA LNP alone or the MDM2 KO LNP alone. The observation of similar synergistic effects in the treatment of different tumor types documents the efficacy of synergic editing for cancer treatment with donor therapeutic mRNA and CRISPR/Cas knockout of pathological cancer genes in a single LNP delivery vector.

### DETAILED DESCRIPTION

Synergic Editing for Treatment of Multigenic Diseases-Whereas significant progress has been made in defining the genetic abnormalities that contribute to the pathogenesis of many diseases, the ability to address these deficiencies through genetic therapies remains limited particularly for multigenic diseases due to the multitude of complementary, amplifying abnormalities that are present which defy amelioration by a single therapy or genetic modification. Cancer is a non-limiting example of a multigenic disease where despite the identification of numerous molecular targets and the development of a variety of genetically targeted treatments and technologies, it remains a leading cause of morbidity and mortality worldwide. While many potentially powerful therapies can be envisioned based upon cancer's molecular pathogenesis, their actual combination is prohibited by either the inefficiencies of current genetic treatments, undruggable targets by conventional methods, development of resistance or by toxicities in normal tissues.

Hence, there is a clear and unmet need for improved genetically targeted therapies particularly for those diseases that have multigenic etiologies and conventionally undruggable components. Disclosed herein are compositions and methods that overcome the deficiencies of current approaches by providing an efficacious in vivo gene editing therapy that concurrently inhibits multiple pathogenic targets in a single treatment while simultaneously expressing therapeutic agents. Importantly, the approach can also effectively address any molecular target including those undruggable by conventional methods. Additionally, and significantly, the disclosed compositions and methods permit the realization of targeted therapy combinations that are too toxic for practical use by conventional treatments and which can reverse treatment resistance. Overall, the disclosed treatments provide an unprecedented ability to attain efficacious synergies and target combinations not previously achievable. The approach is descriptively named “synergic editing” for which non-limiting illustrative embodiments are described below that concurrently disrupt the function of pathogenic genes and simultaneously deliver and express therapeutic nucleic acids.

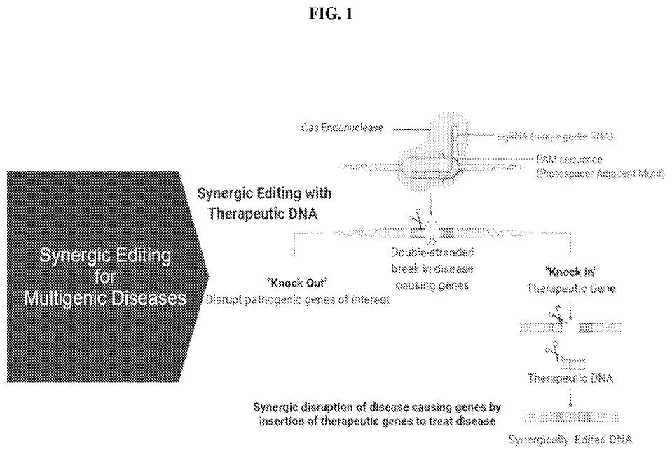

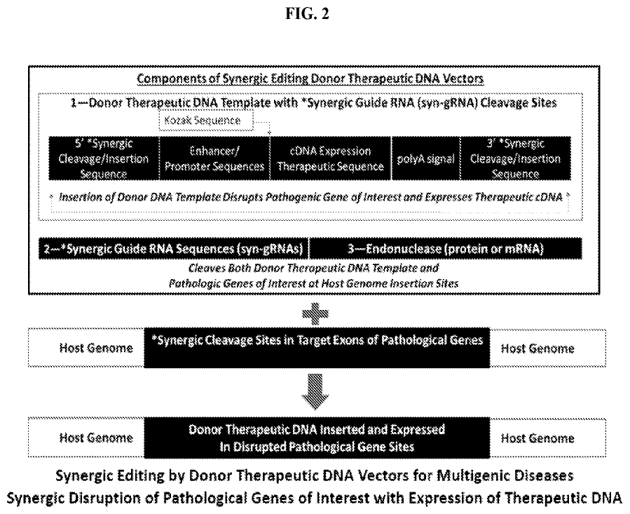

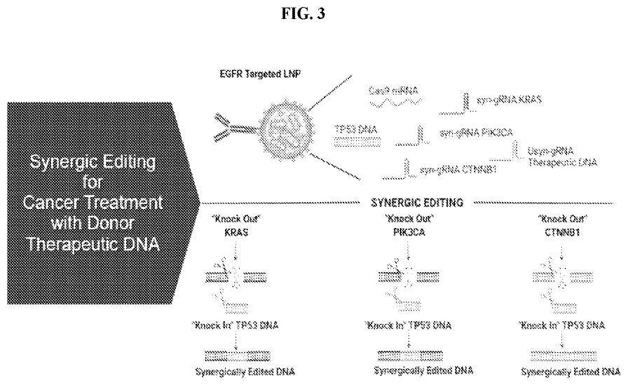

A representative embodiment of synergic editing for multigenic diseases with Donor Therapeutic DNA Vectors is shown generally in FIG. 1 and FIG. 2 which depict synergic disruption of pathological genes of interest by the insertion and expression of therapeutic DNA at those pathogenic gene genomic sites to treat multigenic diseases. FIG. 3 diagrams representative synergic editing for cancer treatment with donor therapeutic DNA vectors. Without the intention of limiting descriptions regarding the multigenic diseases, genes or vectors that can be targeted/utilized, these figures provide non-limiting depictions of representative embodiments of the disclosure. In one embodiment, vectors for multigenic disease treatment (Donor Therapeutic DNA Vectors) are provided comprising a disease cell-targeting vector (e.g., a lipid nanoparticle (LNP)) incorporating a targeting moiety containing: 1) a DNA expression sequence (“Donor Therapeutic DNA sequence”) which contains 5′ and/or 3′ flanking sequences that include an endonuclease cleavage site (a “synergic cleavage/insertion sequence”) that can be cleaved by a “synergic guide RNA” (“syn-gRNA”)-endonuclease complex designed to match in sequence and concurrently cleave a pathological gene genomic site sequence resulting in simultaneous disruption of the pathological gene and the insertion/expression of the Donor Therapeutic DNA sequence (collectively, a “Donor Therapeutic DNA Template with Synergic Guide RNA (syn-gRNA) Cleavage Sites”); 2) guide RNAs (synergic guide RNA (syn-gRNA) sequence(s)) designed to complex with an endonuclease to both cleave/disrupt pathological genes of interest and to cleave the matched 5′ and/or 3′ flanking sequences in the Donor Therapeutic DNA Template facilitating its integration and expression at the disrupted pathogenic gene site; and 3) an endonuclease that auto-complexes with the syn-gRNA. The endonuclease may be provided as either an mRNA expression sequence or as a pre-complexed ribonucleoprotein (RNP) with the syn-gRNAs.

An illustrative embodiment of Synergic Editing for Cancer Treatment with Donor Therapeutic DNA is further diagrammed in FIG. 3. Without the intention of any limitation, this figure provides a non-limiting depiction of representative Donor Therapeutic DNA Vectors for multigenic cancer treatment comprising a cancer cell targeting vector (e.g., a lipid nanoparticle (LNP)) containing a tumor targeting moiety (exemplified by, but not limited to, anti-epidermal growth factor receptor antibody), donor therapeutic DNA (exemplified by, but not limited to, tumor suppressor, pro-apoptotic, and/or immune stimulatory DNAs); synergic guide RNAs (syn-gRNAs), and an endonuclease (as described in FIG. 2) that disrupt pathological cancer genes of interest (exemplified by, but not limited to, oncogenes, anti-apoptotic, and immune suppressive genes) and concurrently express a therapeutic cDNA. In some embodiments, a “universal” synergic cleavage/insertion sequence may be used in the Donor Therapeutic DNA template necessitating the addition of “universal synergic guide RNA” (“Usyn-gRNA”) sequence(s) to the vector. When utilized, the Usyn-gRNA-endonuclease complex cleaves the Donor Therapeutic DNA Template facilitating its integration into the pathogenic cancer genomic sites which are cleaved by the syn-gRNA-endonuclease complexes.

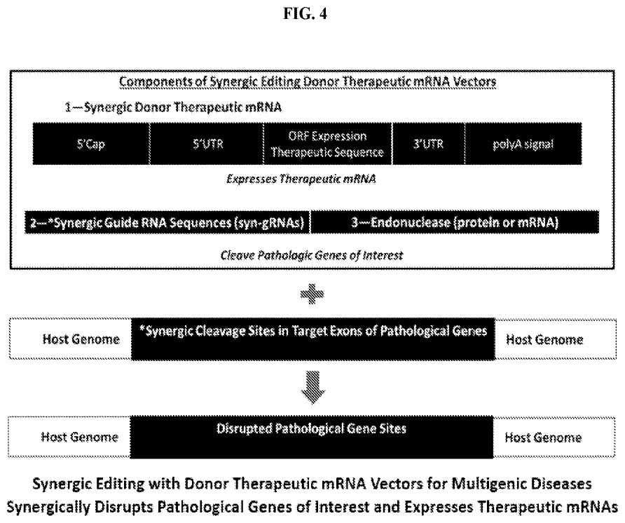

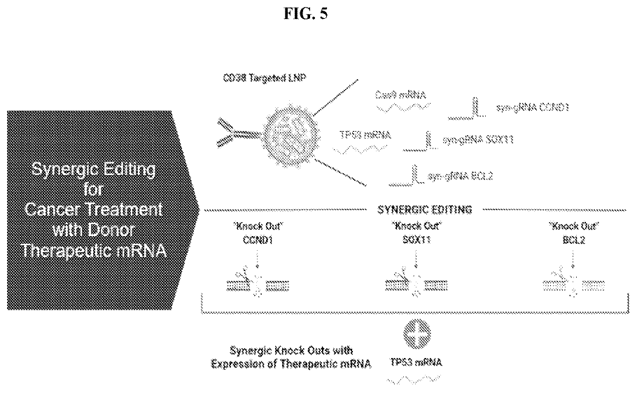

Another illustrative embodiment is Synergic Editing for Multigenic Diseases with Donor Therapeutic mRNA diagrammed in FIG. 4 which depicts the vector compositions and general methods for synergic editing to treat multigenic diseases with donor therapeutic mRNA vectors that synergically disrupt pathological genes of interest and express a therapeutic mRNA. Synergic Editing for Cancer Treatment with Donor Therapeutic mRNA is diagrammed in FIG. 5 and depicts the general vector compositions and methods for synergic editing to treat multigenic cancers with donor therapeutic mRNA Vectors. Without the intention of any limitation, FIG. 5 provides a non-limiting depiction of representative Donor Therapeutic mRNA Vectors for multigenic cancer treatment comprising a cancer cell targeting vector (e.g., lipid nanoparticle (LNP)) containing a tumor targeting moiety (exemplified by, but not limited to, anti-CD38 antibody), therapeutic mRNA (exemplified by, but not limited to, tumor suppressor, pro-apoptotic, and/or immune stimulatory mRNAs); syn-gRNA/single guide RNA and an endonuclease (as described in FIG. 4) that disrupt pathological cancer genes of interest (exemplified by, but not limited to, oncogenes, anti-apoptotic, and immune suppressive genes) and synergically express a therapeutic mRNA.

### Components of Synergic Editing Donor Therapeutic DNA and RNA Vectors-Syn-gRNAs

In embodiments of Synergic Editing with Therapeutic Donor DNA Vectors, syn-gRNAs complex with an endonuclease and serve the dual purposes of disrupting pathogenic genes of interest in the host genome and cleaving the insertion facilitating Synergic Cleavage/Insertion Sequences in the Donor Therapeutic DNA Template (see FIG. 2 and FIG. 3). In embodiments of Synergic Editing with Donor Therapeutic mRNA Vectors, syn-gRNAs/single guide RNAs complex with an endonuclease to disrupt pathogenic genes of interest in the host genome while synergic treatment efficacy is provided by therapeutic mRNA sequences in the vectors (See FIG. 4 and FIG. 5). For multigenic cancer therapy applications, the pathologic genes of interest to be disrupted include, but are not limited to, oncogenes, mutated tumor suppressor genes, angiogenic genes, immune suppressive genes, anti-apoptotic genes, and therapy resistance genes. Specific examples of these types of pathogenic cancer genes include, but are not limited to, HDM2/HDM4/HDMX, BCL2, BCL-XL, mutated p53, BIRC5/Survivin, AKT, PLK1, E2F1, CMYC, NFKappaB, CDNKRAS, FOS, JUN, JAK, STAT, PKA, CREB, TGFB, Beta-Catenin, IL10, PD1, PDL1, MET, AXL, SRC, RAS, PIK3, PGP2, GST, MRP1, and BCRP/ABCG2.

A non-limiting list of pathogenic cancer genes for disruption by synergic editing with either donor therapeutic DNA or mRNA vectors and non-limiting exemplary syn-gRNA/single guide RNA sequences for use with a Cas9 endonuclease are listed in Table 1. The representative syn-gRNAs/single guide RNAs depicted in Table 1 reflect the 5′ (+ strand) 20 bp protospacer sequence followed by PAM (NGG) for each syn-gRNA/single guide RNA target. The selected pathologic gene disruption sites in the host human genome are chosen in an early exon that is common to all known splicing isoforms, such that induced out-of-frame indels lead to premature stop codons in later exon(s) that are not the last exon.

TABLE 1
Non-Limiting List of Representative Pathogenic Human Cancer Genes for
Disruption by Synergic Editing and Non-Limiting Examples of syn-gRNA/single guide
RNA Sequences

| | Gene | |
|---|---|---|
| | ID | Representative Designed |
| Gene Name | NCBI | syn-gRNAs/single guide RNAs for use with Cas9 |
| MDM2* | 4193 | AACTTCAAAAGCAATGGCTINGG (SEQ ID NO: 1); |
| | | TTGAAGTTATTAAAGTCTGTNGG (SEQ ID NO: 2); |
| | | GCTGGTGTAAGTGAACATTCNGG (SEQ ID NO: 3); |
| | | TTGTTTTAGGATCTTGATGCNGG (SEQ ID NO: 4) |
| MDM4 | 4194 | CTCTCCTGGACAAATCAATCNGG (SEQ ID NO: 5); |
| | | AGATGTTGAACACTGAGCAGNGG (SEQ ID NO: 6) |
| BCL2 | 596 | GTCCCGGGATGCGGCTGGATNGG (SEQ ID NO: 7); |
| | | CATCCCGGGACCCGGTCGCCNGG (SEQ ID NO: 8) |
| BCL2L1/BCL-XL | 598 | GCAGACAGCCCCGCGGTGAANGG (SEQ ID NO: 9); |
| | | GAGACCCCCAGTGCCATCAANGG (SEQ ID NO: 10) |
| AKT1 | 207 | GAAGGTGCGTTCGATGACAGNGG (SEQ ID NO: 11); |
| | | TGATGAAGACGGAGCGGCCCNGG (SEQ ID NO: 12) |
| PLK1 | 5347 | CAATTTGCCGTAGGTAGTATNGG (SEQ ID NO: 13); |
| | | CGTAGGTAGTATCGGGCCTCNGG (SEQ ID NO: 14) |
| BIRC5/Survivin | 332 | CCCTTGCAGATGGCCGAGGCNGG (SEQ ID NO: 15); |
| | | GCTCGTTCTCAGTGGGGCAGNGG (SEQ ID NO: 16) |
| MCL1 | 4170 | CCAAAAGTCGCCCTCCCGGGNGG (SEQ ID NO 17); |
| | | AAGTCGCCCTCCCGGGCGGGNGG (SEQ ID NO: 18) |
| E2F1 | 1869 | TTTCGCCACAGGTGAAGCGGNGG (SEQ ID NO: 19); |
| | | AGTACCTGGCCGAGAGCAGTNGG (SEQ ID NO: 20) |
| MYC | 4609 | ATATCCTCGCTGGGCGCCGGNGG (SEQ ID NO: 21); |
| | | GTCGAGGTCATAGTTCCTGTNGG (SEQ ID NO: 22) |
| NFKappaB/NFKB1 | 4790 | GCTCTAATATTTGAAGGTATNGG (SEQ ID NO: 23); |
| | | TAGGTTGCTCTAATATTTGANGG (SEQ ID NO: 24) |
| KRAS | 3845 | TCTCGACACAGCAGGTCAAGNGG (SEQ ID NO: 25); |
| | | CAATGAGGGACCAGTACATGNGG (SEQ ID NO: 26) |
| FOS | 2353 | CAGGTCCGGACTGGTCGAGANGG (SEQ ID NO: 27); |
| | | CGTGGGAATGAAGTTGGCACNGG (SEQ ID NO: 28) |
| JUN | 3725 | TCGTTCCTCCCGTCCGAGAGNGG (SEQ ID NO: 29); |
| | | CCATAAGGTCCGCTCTCGGANGG (SEQ ID NO: 30); |
| | | GTCATGAACCACGTTAACAGNGG (SEQ ID NO: 31); |
| | | GGAGAGAATCGCCCGGCTGGNGG (SEQ ID NO: 32) |
| JAK2 | 3717 | CTGAGCGAACAGTTTCCATCNGG (SEQ ID NO: 33); |
| | | AGTTCTTCTTTGTCCCACTGNGG (SEQ ID NO: 34) |
| STAT | | |
| STAT3 | 6774 | CATTCGACTCTTGCAGGAAGNGG (SEQ ID NO: 35); |
| | | GCAGGAAGCGGCTATACTGCNGG (SEQ ID NO: 36) |
| STAT5/STAT5A | 6776 | TCTGGGACATGGCGTCAACCNGG (SEQ ID NO: 37); |
| | | GCGTCAACCAGGATCCCAGCNGG (SEQ ID NO: 38) |
| PKA | | |
| CREB/CREB1 | 1385 | TAACTGATTCCCAAAAGCGANGG (SEQ ID NO: 39); |
| | | AGCGAAGGGAAATTCTTTCANGG (SEQ ID NO: 40) |
| CCND1 | 595 | GGGGGCCACTTGCATGTTCGNGG (SEQ ID NO: 41); |
| | | TGCACAGCTTCTCGGCCGTCNGG (SEQ ID NO: 42) |
| CCND2 | 894 | GAATTACCTGGACCGTTTCTNGG (SEQ ID NO: 43); |
| | | ACCTGGACCGTTTCTTGGCTNGG (SEQ ID NO: 44) |
| CCND3 | 896 | CCGCCATCCATGATCGCCACNGG (SEQ ID NO: 45); |
| | | ATGATCGCCACGGGCAGCATNGG (SEQ ID NO: 46) |
| SOX11 | 6664 | AAGCGGAGAGCAACCTGCCCNGG (SEQ ID NO: 47); |
| | | CGAATTCATGGCTTGCAGCCNGG (SEQ ID NO: 48) |
| BTK | 695 | GCCCTTCATCATATACAACCNGG (SEQ ID NO: 49); |
| | | ACCCAGGTTGTATATGATGANGG (SEQ ID NO: 50) |
| CDK4 | 1019 | CTTGATCTCCCGGTCAGTTCNGG (SEQ ID NO: 51); |
| | | CCTTGATCTCCCGGTCAGTTNGG (SEQ ID NO: 52) |
| CDK2 | 1017 | GGTGCAGAAATTCAAAAACCNGG (SEQ ID NO: 53); |
| | | CATTCCTCTTCCCCTCATCANGG (SEQ ID NO: 54) |
| CCNE1 | 898 | CGAGGCTTGCACGTTGAGTTNGG (SEQ ID NO: 55); |
| | | ACGTTGAGTTTGGGTAAACCNGG (SEQ ID NO: 56) |
| TGFB/TGFB1 | 7040 | CGGGAGAGCAACACGGGTTCNGG (SEQ ID NO: 57); |
| | | CGGGTTCAGGTACCGCTTCTNGG (SEQ ID NO: 58) |
| Beta- | 1499 | TCCCACTAATGTCCAGCGTTNGG (SEQ ID NO: 59); |
| Catenin/CTNNB1 | | GCCAAACGCTGGACATTAGTNGG (SEQ ID NO: 60) |
| IL10 | 3586 | CAAGGCGCATGTGAACTCCCNGG (SEQ ID NO: 61); |
| | | GGCGCATGTGAACTCCCTGGNGG (SEQ ID NO: 62) |
| MET | 4233 | CCAAATAGTGCACCCCTTGANGG (SEQ ID NO: 63); |
| | | GCTGGGACTTTGGATTTCGGNGG (SEQ ID NO: 64) |
| AXL | 558 | AACATTAGTGCTACGCGGAANGG (SEQ ID NO: 65); |
| | | CGCGTAGCACTAATGTTCTCNGG (SEQ ID NO: 66) |
| SRC | 6714 | CTATGACTATGAGTCTAGGANGG (SEQ ID NO: 67); |
| | | GCTCGCCTTTCTTGAAGGACNGG (SEQ ID NO: 68) |
| PIK3 | 5294 | CAACCAGCTTCGCAAGCAATNGG (SEQ ID NO: 69); |
| | | TGGGCATTTCTGCTCGAACCNGG (SEQ ID NO: 70) |
| PGP2 | 5243 | TTGGCTTGACAAGTTGTATANGG (SEQ ID NO: 71); |
| | | GCTGCCATCATCCATGGGGCNGG (SEQ ID NO: 72) |
| GST/GSTP1 | 2950 | ATCTGCCAGCAGCATGCGCANGG (SEQ ID NO: 73); |
| | | ACCGTGGAGACGTGGCAGGANGG (SEQ ID NO: 74) |
| MRP1/ABCC1 | 4363 | TTTGCTGTGGATCGTCTGCTNGG (SEQ ID NO: 75); |
| | | TTTTGCTGTGGATCGTCTGCNGG (SEQ ID NO: 76) |
| BCRP/ABCG2 | 9429 | ACATCTCCAGATAATCCACTNGG (SEQ ID NO: 77); |
| | | GCTGCAAGGAAAGATCCAAGNGG (SEQ ID NO: 78) |

*An additional MDM2 single guide RNA is the sequence corresponding to position 68820351 positive strand CTTGGTAGTAGTCAATCAGC (SEQ ID NO: 81) with PAM AGG from Integrated DNA Technologies (IDT) Design ID: Hs.Cas9.MDM2.1.AA.

Additional pathogenic cancer genes for disruption include, but are not limited to, RAB25, EVI1, EIF5A2, PRKC1, MYK, EGFR, NOTCH3, ERBB2, HER2, HER3, HER4, PIK3R1, CCNE1, AKT2, and AURKA, KIT, RAS, RAF, MEK, MAPK, ERK, PI3K, Akt, MET, mTORC1, mTOR, IGF-1R, IGF-2R, HIF1-alpha, and SMAD4.

Endonucleases (Protein or mRNA)

In certain embodiments, the endonuclease employed in synergic editing with either donor therapeutic DNA or mRNA vectors is a CRISPR-associated endonuclease (Cas). The design of the syn-gRNA/single guide RNA PAMs will vary depending upon the CRISPR-associated (Cas) endonuclease selected for incorporation in the Donor Therapeutic DNA or mRNA vectors. In certain embodiments, Cas endonucleases and their PAM sequences for use in synergic editing with either donor therapeutic DNA or mRNA vectors are described further below and are listed in Table 2. The Cas endonuclease may be provided in the vector as either an expression mRNA or complexed with a syn-gRNA/single guide RNA as a ribonucleoprotein (RNP) (See FIGS. 1-5). In certain embodiments, the CRISPR-associated endonuclease is Cas9, Cas12a, Cas12b, Cas12e, Cas13a, Cas13b, Cas14, Cas-theta, CasX, or CasY. A partial list of Cas endonucleases and their protospacer adjacent motifs include but are not limited to those listed in Table 2. Cas endonucleases with nuclear localization signal sequences (NLS) may be provided as either an mRNA encoding the Cas endonuclease (e.g., a CleanCap Cas9 mRNA (modified) custom manufactured by TriLink BioTechnologies Inc) or complexed with a syn-gRNA/single guide RNA as a ribonucloprotein (RNP) (e.g., custom manufactured by Aldevron Inc).

TABLE 2
Representative Cas endonucleases and their protospacer adjacent motifs
(PAM)

| CRISPR | Organism Isolated | |
|---|---|---|
| Nucleases | From | PAM Sequence (5′ to 3′) |
| SpCas9* | Streptococcus pyogenes | NGG |
| SaCas9 | Staphylococcus aureus | NGRRT or NGRRN |
| NmeCas9 | Neisseria meningitidis | NNNNGATT |
| CjCas9 | Campylobacter jejuni | NNNNRYAC |
| StCas9 | Streptococcus | NNAGAAW |
| | thermophilus | |
| LbCpf1 | Lachnospiraceae bacterium | TTTV |
| (Cas12a) | | |
| AsCpf1 | Acidaminococcus sp. | TTTV |
| (Cas12a) | | |
| AacCas12b | Alicyclobacillus | TTN |
| | acidiphilus | |
| BhCas12b v4 | Bacillus hisashii | ATTN, TTTN and GTTN |
| Cas14 | Uncultivated archea | T-rich PAM sequences, e.g., TTTA for dsDNA |
| | | cleavage, no PAM sequence requirement for ssDNA |

*With respect to CRISPR/Cas endonucleases which may be incorporated in vectors (e.g.,
LNPs) in either ribonucleoprotein or mRNA forms, a representative protein sequence for high
fidelity (HiFi) Cas9 endonuclease is listed below:
DKKYSIGLDIGTNSVGWAVITDEYKVPSKKFKVLGNTDRHSIKKNLIGALLFDSGETAE
ATRLKRTARRRYTRRKNRICYLQEIFSNEMAKVDDSFFHRLEESFLVEEDKKHERHPIFG
NIVDEVAYHEKYPTIYHLRKKLVDSTDKADLRLIYLALAHMIKFRGHFLIEGDLNPDNS
DVDKLFIQLVQTYNQLFEENPINASGVDAKAILSARLSKSRRLENLIAQLPGEKKNGLFG
NLIALSLGLTPNFKSNEDLAEDAKLQLSKDTYDDDLDNLLAQIGDQYADLFLAAKNLSD
AILLSDILRVNTEITKAPLSASMIKRYDEHHQDLTLLKALVRQQLPEKYKEIFFDQSKNG
YAGYIDGGASQEEFYKFIKPILEKMDGTEELLVKLNREDLLRKQRTFDNGSIPHQIHLGE
LHAILRRQEDFYPFLKDNREKIEKILTFRIPYYVGPLARGNSRFAWMTRKSEETITPWNF
EEVVDKGASAQSFIERMTNFDKNLPNEKVLPKHSLLYEYFTVYNELTKVKYVTEGMRK
PAFLSGEQKKAIVDLLFKTNRKVTVKQLKEDYFKKIECFDSVEISGVEDRFNASLGTYH
DLLKIIKDKDFLDNEENEDILEDIVLTLTLFEDREMIEERLKTYAHLEDDKVMKQLKRRR
YTGWGRLSRKLINGIRDKQSGKTILDFLKSDGFANANFMQLIHDDSLTFKEDIQKAQVS
GQGDSLHEHIANLAGSPAIKKGILQTVKVVDELVKVMGRHKPENIVIEMARENQTTQK
GQKNSRERMKRIEEGIKELGSQILKEHPVENTQLQNEKLYLYYLQNGRDMYVDQELDI
NRLSDYDVDHIVPQSFLKDDSIDNKVLTRSDKNRGKSDNVPSEEVVKKMKNYWRQLL
NAKLITQRKFDNLTKAERGGLSELDKAGFIKRQLVETRQITKHVAQILDSRMNTKYDEN
DKLIREVKVITLKSKLVSDFRKDFQFYKVREINNYHHAHDAYLNAVVGTALIKKYPKLE
SEFVYGDYKVYDVRKMIAKSEQEIGKATAKYFFYSNIMNFFKTEITLANGEIRKRPLIET
NGETGEIVWDKGRDFATVRKVLSMPQVNIVKKTEVQTGGFSKESILPKRNSDKLIARKK
DWDPKKYGGFDSPTVAYSVLVVAKVEKGKSKKLKSVKELLGITIMERSSFEKNPIDFLE
AKGYKEVKKDLIIKLPKYSLFELENGRKRMLASAGELQKGNELALPSKYVNFLYLASH
YEKLKGSPEDNEQKQLFVEQHKHYLDEDIEQISEFSKRVILADANLDKVLSAYNKHRDK
PIREQAENIIHLFTLTNLGAPAAFKYFDTTIDRKRYTSTKEVLDATLIHQSITGLYETRIDL
SQLGGD (SEQ ID NO: 82)
*An additional Cas9 mRNA sequence is a modified mRNA sold by TriLink Biotechnologies
(Catalog Number L-7206) provided below:
AUGGCCCCCAAGAAGAAGCGGAAGGUGGGCAUCCACGGCGUGCCCGCCGCCGACA
AGAAGUACAGCAUCGGCCUGGACAUCGGCACCAACAGCGUGGGCUGGGCCGUGA
UCACCGACGAGUACAAGGUGCCCAGCAAGAAGUUCAAGGUGCUGGGCAACACCG
ACCGGCACAGCAUCAAGAAGAACCUGAUCGGCGCCCUGCUGUUCGACAGCGGCGA
GACCGCCGAGGCCACCCGGCUGAAGCGGACCGCCCGGCGGCGGUACACCCGGCGG
AAGAACCGGAUCUGCUACCUGCAGGAGAUCUUCAGCAACGAGAUGGCCAAGGUG
GACGACAGCUUCUUCCACCGGCUGGAGGAGAGCUUCCUGGUGGAGGAGGACAAG
AAGCACGAGCGGCACCCCAUCUUCGGCAACAUCGUGGACGAGGUGGCCUACCACG
AGAAGUACCCCACCAUCUACCACCUGCGGAAGAAGCUGGUGGACAGCACCGACAA
GGCCGACCUGCGGCUGAUCUACCUGGCCCUGGCCCACAUGAUCAAGUUCCGGGGC
CACUUCCUGAUCGAGGGCGACCUGAACCCCGACAACAGCGACGUGGACAAGCUGU
UCAUCCAGCUGGUGCAGACCUACAACCAGCUGUUCGAGGAGAACCCCAUCAACGC
CAGCGGCGUGGACGCCAAGGCCAUCCUGAGCGCCCGGCUGAGCAAGAGCCGGCGG
CUGGAGAACCUGAUCGCCCAGCUGCCCGGCGAGAAGAAGAACGGCCUGUUCGGC
AACCUGAUCGCCCUGAGCCUGGGCCUGACCCCCAACUUCAAGAGCAACUUCGACC
UGGCCGAGGACGCCAAGCUGCAGCUGAGCAAGGACACCUACGACGACGACCUGG
ACAACCUGCUGGCCCAGAUCGGCGACCAGUACGCCGACCUGUUCCUGGCCGCCAA
GAACCUGAGCGACGCCAUCCUGCUGAGCGACAUCCUGCGGGUGAACACCGAGAUC
ACCAAGGCCCCCCUGAGCGCCAGCAUGAUCAAGCGGUACGACGAGCACCACCAGG
ACCUGACCCUGCUGAAGGCCCUGGUGCGGCAGCAGCUGCCCGAGAAGUACAAGG
AGAUCUUCUUCGACCAGAGCAAGAACGGCUACGCCGGCUACAUCGACGGCGGCGC
CAGCCAGGAGGAGUUCUACAAGUUCAUCAAGCCCAUCCUGGAGAAGAUGGACGG
CACCGAGGAGCUGCUGGUGAAGCUGAACCGGGAGGACCUGCUGCGGAAGCAGCG
GACCUUCGACAACGGCAGCAUCCCCCACCAGAUCCACCUGGGCGAGCUGCACGCC
AUCCUGCGGCGGCAGGAGGACUUCUACCCCUUCCUGAAGGACAACCGGGAGAAG
AUCGAGAAGAUCCUGACCUUCCGGAUCCCCUACUACGUGGGCCCCCUGGCCCGGG
GCAACAGCCGGUUCGCCUGGAUGACCCGGAAGAGCGAGGAGACCAUCACCCCCUG
GAACUUCGAGGAGGUGGUGGACAAGGGCGCCAGCGCCCAGAGCUUCAUCGAGCG
GAUGACCAACUUCGACAAGAACCUGCCCAACGAGAAGGUGCUGCCCAAGCACAGC
CUGCUGUACGAGUACUUCACCGUGUACAACGAGCUGACCAAGGUGAAGUACGUG
ACCGAGGGCAUGCGGAAGCCCGCCUUCCUGAGCGGCGAGCAGAAGAAGGCCAUC
GUGGACCUGCUGUUCAAGACCAACCGGAAGGUGACCGUGAAGCAGCUGAAGGAG
GACUACUUCAAGAAGAUCGAGUGCUUCGACAGCGUGGAGAUCAGCGGCGUGGAG
GACCGGUUCAACGCCAGCCUGGGCACCUACCACGACCUGCUGAAGAUCAUCAAGG
ACAAGGACUUCCUGGACAACGAGGAGAACGAGGACAUCCUGGAGGACAUCGUGC
UGACCCUGACCCUGUUCGAGGACCGGGAGAUGAUCGAGGAGCGGCUGAAGACCU
ACGCCCACCUGUUCGACGACAAGGUGAUGAAGCAGCUGAAGCGGCGGCGGUACA
CCGGCUGGGGCCGGCUGAGCCGGAAGCUGAUCAACGGCAUCCGGGACAAGCAGA
GCGGCAAGACCAUCCUGGACUUCCUGAAGAGCGACGGCUUCGCCAACCGGAACUU
CAUGCAGCUGAUCCACGACGACAGCCUGACCUUCAAGGAGGACAUCCAGAAGGCC
CAGGUGAGCGGCCAGGGCGACAGCCUGCACGAGCACAUCGCCAACCUGGCCGGCA
GCCCCGCCAUCAAGAAGGGCAUCCUGCAGACCGUGAAGGUGGUGGACGAGCUGG
UGAAGGUGAUGGGCCGGCACAAGCCCGAGAACAUCGUGAUCGAGAUGGCCCGGG
AGAACCAGACCACCCAGAAGGGCCAGAAGAACAGCCGGGAGCGGAUGAAGCGGA
UCGAGGAGGGCAUCAAGGAGCUGGGCAGCCAGAUCCUGAAGGAGCACCCCGUGG
AGAACACCCAGCUGCAGAACGAGAAGCUGUACCUGUACUACCUGCAGAACGGCC
GGGACAUGUACGUGGACCAGGAGCUGGACAUCAACCGGCUGAGCGACUACGACG
UGGACCACAUCGUGCCCCAGAGCUUCCUGAAGGACGACAGCAUCGACAACAAGG
UGCUGACCCGGAGCGACAAGAACCGGGGCAAGAGCGACAACGUGCCCAGCGAGG
AGGUGGUGAAGAAGAUGAAGAACUACUGGCGGCAGCUGCUGAACGCCAAGCUGA
UCACCCAGCGGAAGUUCGACAACCUGACCAAGGCCGAGCGGGGCGGCCUGAGCGA
GCUGGACAAGGCCGGCUUCAUCAAGCGGCAGCUGGUGGAGACCCGGCAGAUCAC
CAAGCACGUGGCCCAGAUCCUGGACAGCCGGAUGAACACCAAGUACGACGAGAA
CGACAAGCUGAUCCGGGAGGUGAAGGUGAUCACCCUGAAGAGCAAGCUGGUGAG
CGACUUCCGGAAGGACUUCCAGUUCUACAAGGUGCGGGAGAUCAACAACUACCA
CCACGCCCACGACGCCUACCUGAACGCCGUGGUGGGCACCGCCCUGAUCAAGAAG
UACCCCAAGCUGGAGAGCGAGUUCGUGUACGGCGACUACAAGGUGUACGACGUG
CGGAAGAUGAUCGCCAAGAGCGAGCAGGAGAUCGGCAAGGCCACCGCCAAGUAC
UUCUUCUACAGCAACAUCAUGAACUUCUUCAAGACCGAGAUCACCCUGGCCAACG
GCGAGAUCCGGAAGCGGCCCCUGAUCGAGACCAACGGCGAGACCGGCGAGAUCG
UGUGGGACAAGGGCCGGGACUUCGCCACCGUGCGGAAGGUGCUGAGCAUGCCCC
AGGUGAACAUCGUGAAGAAGACCGAGGUGCAGACCGGCGGCUUCAGCAAGGAGA
GCAUCCUGCCCAAGCGGAACAGCGACAAGCUGAUCGCCCGGAAGAAGGACUGGG
ACCCCAAGAAGUACGGCGGCUUCGACAGCCCCACCGUGGCCUACAGCGUGCUGGU
GGUGGCCAAGGUGGAGAAGGGCAAGAGCAAGAAGCUGAAGAGCGUGAAGGAGCU
GCUGGGCAUCACCAUCAUGGAGCGGAGCAGCUUCGAGAAGAACCCCAUCGACUU
CCUGGAGGCCAAGGGCUACAAGGAGGUGAAGAAGGACCUGAUCAUCAAGCUGCC
CAAGUACAGCCUGUUCGAGCUGGAGAACGGCCGGAAGCGGAUGCUGGCCAGCGC
CGGCGAGCUGCAGAAGGGCAACGAGCUGGCCCUGCCCAGCAAGUACGUGAACUU
CCUGUACCUGGCCAGCCACUACGAGAAGCUGAAGGGCAGCCCCGAGGACAACGAG
CAGAAGCAGCUGUUCGUGGAGCAGCACAAGCACUACCUGGACGAGAUCAUCGAG
CAGAUCAGCGAGUUCAGCAAGCGGGUGAUCCUGGCCGACGCCAACCUGGACAAG
GUGCUGAGCGCCUACAACAAGCACCGGGACAAGCCCAUCCGGGAGCAGGCCGAGA
ACAUCAUCCACCUGUUCACCCUGACCAACCUGGGCGCCCCCGCCGCCUUCAAGUA
CUUCGACACCACCAUCGACCGGAAGCGGUACACCAGCACCAAGGAGGUGCUGGAC
GCCACCCUGAUCCACCAGAGCAUCACCGGCCUGUACGAGACCCGGAUCGACCUGA
GCCAGCUGGGCGGCGACAGCGGCGGCAAGCGGCCCGCCGCCACCAAGAAGGCCGG
CCAGGCCAAGAAGAAGAAGGGCAGCUACCCCUACGACGUGCCCGACUACGCCUGA
(SEQ ID NO: 83)

Donor Therapeutic DNA Templates with Syn-gRNA Cleavage Sites

In particular embodiments of Synergic Editing with Therapeutic Donor DNA Vectors as diagrammed in FIG. 2, the Donor Therapeutic DNA Template includes Synergic Cleavage/Insertion Sequences (5′ or 5′ and 3′ positions), Enhancer/Promoter Sequences, cDNA Expression Therapeutic Sequence and a polyA signal which encompass a translation initiation sequence, a start codon, a termination codon, and a transcription termination sequence.

Donor Therapeutic DNA templates for synergic editing contain cleavage/insertion sequences at the 5′ or 5′ and 3′ positions that can be cleaved by syn-gRNA-Cas complexes as they include the associated Cas PAM. The syn-gRNAs in the vector result in the cleavage of both the pathogenic gene of interest and the synergic cleavage/insertion sequence in the Donor Therapeutic DNA (See FIGS. 2 and 3). These simultaneous cleaving reactions are synergic and result in the disruption of the pathogenic gene of interest and insertion and expression of the Donor Therapeutic DNA at the pathogenic genes of interest sites in the genome. In other embodiments, the donor therapeutic DNA sequence has further dual 5′ and 3′ homology arms, or single 5′ homology arms with the pathological gene of interest insertion site to facilitate integration “knock-in” of the donor therapeutic expression DNA into the host genome (See FIG. 2 and FIG. 3).

Donor Therapeutic DNA with “Universal” Synergic Cleavage/Insertion Sequences at the 5′ or 5′ and 3′ Positions

In certain embodiments, as noted above, “universal” synergic cleavage/insertion sequences may be used in the Donor Therapeutic DNA template in concert with the addition of universal synergic guide RNAs (Usyn-gRNA) to the vector. When utilized, the Usyn-gRNA/Cas complex cleaves the Donor Therapeutic DNA Template facilitating its integration into the pathogenic cancer genomic sites which are cleaved by the syn-gRNA/Cas endonuclease complexes.

In contrast to the standard synergic cleavage/insertion sequences described above which correspond to the disruption sequences of the pathogenic genes of interest, “Universal” synergic cleavage/insertion sequences in the Donor Therapeutic DNA template are designed to have minimal and ideally no sequence homology in the host genome. Rather, “Universal” synergic cleavage/insertion sequences in the Donor Therapeutic DNA template contain a unique sequence that can be cleaved by a separate matched Universal syn-gRNA (Usyn-gRNA) that is included in the Donor Therapeutic DNA Vector to cleave the Donor Therapeutic DNA template facilitating its integration into pathogenic gene sites of interest which are cleaved by their separate matched syn-gRNAs.

Algorithm for Generating Universal Synergic Cleavage/Insertion Sequences and their Matched Usyn-gRNAs

In certain embodiments, an algorithm is utilized to generate Universal Synergic Cleavage/Insertion Sequences which are designed to have minimal and ideally no sequence homology in the host genome to be edited. The first step in the algorithm is to select the Cas endonuclease and to generate candidate Usyn-gRNA sequences of optimal length that have no sequence identity in the host genome. Further characteristics of a Usyn-gRNA are its ability to be cleaved when annealed to the PAM sequences associated with the chosen Cas endonuclease and to have minimal off-target effects in the host genome characterized by few to no mismatch cleavage sites in the host genome. An algorithm for designing a Usyn-gRNA begins with a candidate sequence of the optimal length for the chosen Cas endonuclease that is not found in the host genome. In a representative example for a Cas9 nuclease to be used in humans, a 20 bp candidate is first identified which has no match in a BLAST search of the human genome. The candidate 20 bp DNA sequence can either be a randomly generated sequence or substituted to be comprised mostly of G and C bases which are typically under-represented in the human genome. Alternatively, a starting candidate sequence can be derived from a non-human genome such as a jellyfish genome or an insect genome such as Drosophila. If a non-human genome such as Drosophila is selected for design initiation, candidate 20 bp starting sequences can be identified by using genes that have no human homologs and subjecting them to a knock-out design program such as the Synthego Knock Out Design web tool at design.synthego.com/#/. These starting sequences are then modified to include efficiency optimizing and exclude sequences known to be suboptimal for the Cas endonuclease selected. For example, it is known that Cas 9 is optimized by incorporating a G at the first place and A or T at the 17th place. Inefficient Cas9 guides end with TTC or TTT or contain only T and C in the last four nucleotides and more than 2 Ts or at least one TT and one T or C (′TT-motif). These modified candidate sequences are then tested for potential off-target effects and target sequence efficacy using programs designed for these purpose such as Off-Spotter (cm.jefferson.edu/Off-Spotter/) or CRISPOR (crispor.tefor.net/). The candidate sequences are ranked by the fewest number of mismatch cleavage sites in the human genome, particularly off-targets that have no mismatches in the 12 bp adjacent to the PAM. Representative results employing the algorithm reveal that the best starting candidate sequences are from non-human genes that have no human homologs. Employing the algorithm described above and starting with the non-human Drosophila gene CG10624 (sinu), the Usyn-gRNA target sequence GGAATCCTCGCGTGCGAAGTcgg (SEQ ID NO: 79) was developed which has only 24 off-targets with up to 5 mismatches and no mismatches in the 12 bp adjacent to the PAM. Another candidate Usyn-gRNA target sequence GAGCTGGACGGCGACGTAAAcgg (SEQ ID NO: 80) from the jelly fish EGFP gene has only 26 off-targets with up to 5 mismatches and no mismatches in the 12 bp adjacent to the PAM. Either of these sequences can be used as “Universal” 5′ or 5′ and 3′ synergic cleavage/insertion sequences in the Donor Therapeutic DNA template along with their corresponding Usyn-gRNAs. The donor therapeutic DNA templates can be incorporated in the vectors as either a supercoiled DNA minicircle or linear plasmid which have neither bacterial origins of replication nor antibiotic resistance genes. The therapeutic DNA minicircle and linear plasmids may have flanking Cas9 cleavage sites that are cut by an incorporated Usyn-gRNA/Cas or syng-RNA complex. Donor Therapeutic DNA templates can be custom manufactured as minicircles (e.g., from PlasmidFactory Inc) or as linear plasmids (e.g., from TriLink BioTechnologies Inc). In other embodiments, the donor therapeutic DNA template may have further dual 5′ and 3′ homology arms, or single 5′ homology arms with the pathological gene of interest insertion site to facilitate integration “knock-in” of the therapeutic expression DNA into the host genome.

Components of Synergic Editing Donor Therapeutic mRNA Vectors

In embodiments of synergic editing with Donor Therapeutic mRNA Vectors as diagrammed in FIGS. 4 and 5, instead of a therapeutic DNA, these vectors employ donor therapeutic mRNA for synergic editing with the syn-gRNAs/single guide RNAs and Cas endonuclease components described above. The synergic donor therapeutic mRNA sequence includes a 5′ Cap, a 5′ untranslated region (UTR), an open reading frame (ORF) of an Expression Therapeutic Sequence, a 3′ UTR, and a poly A signal (see FIG. 3). In representative, non-limiting applications for cancer treatments, the synergic editing donor therapeutic mRNAs encode one or more of the categories and/or specific therapeutic genes listed in Table 3. The therapeutic mRNAs may be custom manufactured (e.g., from TriLink BioTechnologies Inc or other commercial vendors) as 5′ capped and polyadenylated mRNAs ready for translation by the ribosome either unmodified, codon optimized, or modified with 5-methoxyuridine to modulate mRNA expression as desired.

A representative, non-limiting list of Donor Therapeutic DNA and Donor Therapeutic mRNA categories with specific examples for cancer treatment by synergic editing is provided in Table 3 below.

TABLE 3
Representative Donor Therapeutic DNAs and mRNAs for Synergic Editing
Cancer Treatment
Tumor Suppressor Genes
TP53*
PTEN
Rb
IL24
APC
ATM
CDKN2A (splicing variants to p16INK4A and p14ARF)
CDKN1B
Pro-Apoptotic Genes
BAX
BAK
BCLX
Enzyme Gene/Prodrug Systems ″Suicide Genes″
Viral Thymidine Kinase--HSV-TK
Cytosine Deaminase (CDA)
Other Enzyme Gene/Prodrug Systems-See Table 4
Immune Stimulating Genes
IL2/IL15 beta/gamma muteins (CD122/132 agonists e.g. F42K)
HSP 70/90
Calreticulin
HMGB1
CGAS
STING1
TLR
IRF3/7
*With respect to TP53 wild type sequences, the therapeutic mRNA may encode
any of the corresponding wild type isoforms listed in the National Center for
Biotechnology Information database for Gene ID 7157. A representative protein
sequence from isoform “a” transcript variant “1” is listed below:
MEEPQSDPSVEPPLSQETFSDLWKLLPENNVLSPLPSQAMDDLMLSPDD
IEQWFTEDPGPDEAPRMPEAAPPVAPAPAAPTPAAPAPAPSWPLSSSVP
SQKTYQGSYGFRLGFLHSGTAKSVTCTYSPALNKMFCQLAKTCPVQL
WVDSTPPPGTRVRAMAIYKQSQHMTEVVRRCPHHERCSDSDGLAPPQ
HLIR VEGNLRVEYLDDRNTFRHSVVVPYEPPEVGSDCTTIHYNYMCNS
SCMGGMNRRPILTIITLEDSSGNLLGRNSFEVRVCACPGRDRRTEEENL
RKKGEPHHELPPGSTKRALPNNTSSSPQPKKKPLDGEYFTLQIRGRERFE
MFRELNEALELKDAQAGKEPGGSRAHSSHLKSKKGQSTSRHKKLMFK
TEGPDSD (SEQ ID NO: 84)
*Another wild type TP53 mRNA sequence is found under Genbank accession
number AF307851:
AUGGAGGAGCCGCAGUCAGAUCCUAGCGUCGAGCCCCCUCUGAGU
CAGGAAACAUUUUCAGACCUAUGGAAACUACUUCCUGAAAACAAC
GUUCUGUCCCCCUUGCCGUCCCAAGCAAUGGAUGAUUUGAUGCUG
UCCCCGGACGAUAUUGAACAAUGGUUCACUGAAGACCCAGGUCCA
GAUGAAGCUCCCAGAAUGCCAGAGGCUGCUCCCCGCGUGGCCCCU
GCACCAGCAGCUCCUACACCGGCGGCCCCUGCACCAGCCCCCUCCU
GGCCCCUGUCAUCUUCUGUCCCUUCCCAGAAAACCUACCAGGGCA
GCUACGGUUUCCGUCUGGGCUUCUUGCAUUCUGGGACAGCCAAGU
CUGUGACUUGCACGUACUCCCCUGCCCUCAACAAGAUGUUUUGCC
AACUGGCCAAGACCUGCCCUGUGCAGCUGUGGGUUGAUUCCACAC
CCCCGCCCGGCACCCGCGUCCGCGCCAUGGCCAUCUACAAGCAGUC
ACAGCACAUGACGGAGGUUGUGAGGCGCUGCCCCCACCAUGAGCG
CUGCUCAGAUAGCGAUGGUCUGGCCCCUCCUCAGCAUCUUAUCCG
AGUGGAAGGAAAUUUGCGUGUGGAGUAUUUGGAUGACAGAAACA
CUUUUCGACAUAGUGUGGUGGUGCCCUAUGAGCCGCCUGAGGUUG
GCUCUGACUGUACCACCAUCCACUACAACUACAUGUGUAACAGUU
CCUGCAUGGGCGGCAUGAACCGGAGGCCCAUCCUCACCAUCAUCA
CACUGGAAGACUCCAGUGGUAAUCUACUGGGACGGAACAGCUUUG
AGGUGCGUGUUUGUGCCUGUCCUGGGAGAGACCGGCGCACAGAGG
AAGAGAAUCUCCGCAAGAAAGGGGAGCCUCACCACGAGCUGCCCC
CAGGGAGCACUAAGCGAGCACUGCCCAACAACACCAGCUCCUCUC
CCCAGCCAAAGAAGAAACCACUGGAUGGAGAAUAUUUCACCCUUC
AGAUCCGUGGGCGUGAGCGCUUCGAGAUGUUCCGAGAGCUGAAU
GAGGCCUUGGAACUCAAGGAUGCCCAGGCUGGGAAGGAGCCAGGG
GGGAGCAGGGCUCACUCCAGCCACCUGAAGUCCAAAAAGGGUCAG
UCUACCUCCCGCCAUAAAAAACUCAUGUUCAAGACAGAAGGGCCU
GACUCAGACAAGGGCUCGAGUCUAGAGGGCCCGCGGUUCGAAGGU
AAGCCUAUCCCUAACCCUCUCCUCGGUCUCGAUUCUACGCGUACC
GGUUAG (SEQ ID NO: 85)

TABLE 4
Representative Enzyme Gene-Prodrug Systems for Donor Therapeutic
DNAs and mRNAs for Synergic Editing Cancer Treatment

| Gene Names and | | | |
|---|---|---|---|
| Enzyme | | | |
| Commission | | | |
| (EC) Codes | Origin | Prodrugs | Released (pro)drugs |
| Carboxyl esterase | Human, rabbit | Irinotecan 7-ethyl- | SN-38 7-ethyl-10- |
| (CE) | | 10-[4-(1- | hydroxy-(20S)- |
| | | piperidino)-1- | camptothecin |
| | | piperidino]- | |
| | | carbonyloxy-(20S)- | |
| | | camptothecin | |
| Carboxypeptidase | Human | MTX-α-peptides | MTX |
| A (CPA) | | | |
| Carboxypeptidase | Pseudomonas | CMDA ZD-2767 | CMBA phenol- |
| G2 (CPG2) EC | str. | Self-immolative | bisiodonitrogen |
| 3.4.22. 12 | | | mustard; alkylating |
| | | | agents, anthracycline |
| | | | antibiotics |
| Cytochrome | Human, rat, | Oxazaphosphorines, | Alkylating agents, |
| P450, Human; | rabbit | ipomeanol, 2- | toxic metabolites, |
| CYP2B1, 2B6, | | aminoanthracene | N-acetyl-benzoquinone |
| 2C8, 2C9, 2C18, | | (2-AA); | imine (NABQI) |
| and 3A Rat: | | acetaminophen | |
| CYP2B1 Rabbit: | | | |
| 4B1 (with or | | | |
| without red- | | | |
| P450) | | | |
| Cytosine | E. coli, yeast | 5-Fluorocytosine | 5-Fluorouracil (5-FU) |
| deaminase (CD) | | (5-FC) | |
| EC 3.5.4.1 (with | | | |
| or without uracil- | | | |
| phosphoribosyl | | | |
| transferase, | | | |
| UPRT) | | | |
| D-Amino acid | Rodhoto rula | D-Alanine | Hydrogen peroxide |
| oxidase (DAAO) | gracilis, (yeast) | | |
| Deoxycytidine | Human | Cytosine | Cytosine arabinoside |
| kinase (dCK), | | arabinoside | monophosphate |
| EC.2.7.1.21 | | | |
| Deoxyribo- | Drosophila | Analogues of | Analogues of |
| nucleotide kinase | melanogaster | pyrimi-dine and | pyrimidine and purine |
| (DmNK) | | purine 2′- | 2′-deoxynucleotide |
| | | deoxynucleosides | monophosphates |
| DT-Diaphorase | Human, rat? | Bioreductive | Reduced forms? |
| (DT-D) | | agents: EO9, etc. | |
| β-Galactosidase | E. coli | Self-immolative | Anthracycline |
| (β-Gal) EC | | prodrugs from | antibiotics |
| 3.2.1.23 | | anthracyclin | |
| | | antibiotics | |
| β-Glucuronidase | Human | Self-immolative | Doxorubicin |
| (β-Glu) | | HM-1826 | |
| β-Lactamase | Bacterial? | Self-immolative | Alkylating |
| (β-L) | | (cephem prodrugs) | agents, Vinca alkaloids, |
| | | | anthracycline |
| | | | antibiotics |
| Methionine-α, | Pseudomonas | Selenomethionine | Methylselenol |
| γ-lyase (MET) | putida | | |
| Multiple drug | Tomato | Acetylated 6-TG, | 6-TG, MTX, cytotoxic |
| activating | | MTX, and other | purines |
| enzyme (MDAE) | | purines | |
| Nitroreductase | E. coli | CB-1954 and | Alkylating agents, |
| (NR) | | analogues; self- | pyrazolidines, |
| | | immolative | enediynes |
| Purine nucleotide | E. coli | Purine nucleosides | 6-Methylpurine, |
| phosphorylase | | | 2-fluoroadenine |
| (PNP), EC2.4.2.1 | | | |
| Thymidine | HSV or V2V | Modified | Monophosphate |
| kinase, (TK), | | pyrimidine | nucleotide analogues |
| EC2.7.1.21 | | nucleosides: GCV, | |
| | | ACV, valacyclovir, | |
| | | etc; FIAU, purine | |
| | | nucleosides | |
| Thymidine | Human | Pyrimidine | 5-Fluorodeoxyuridine |
| phosphorylase | | analogues, | monophosphate |
| (TP), EC2.4.2.4 | | 5′-DFUR | (5-FdRMP) |
| Xanthine-guanine | E. coli | 6-Thiopurines | 6-Thiopurine |
| phosphoribbosyl | | | nucleoside |
| transferase | | | |
| (XGPT) | | | |

In some embodiments, additional genes in the tumor suppressor category include, but are not limited to, ARHI, RASSF1A, DLEC1, SPARC, DAB2, PLAGL1, RPS6KA2, PTEN, OPCML, BRCA2, ARL11, WWOX, TP53, DPH1, BRCA1, mTORC2, and PEG3. In certain embodiments, donor therapeutic DNA or donor therapeutic mRNA vector components for synergic editing may encode and express genes that are silenced by hypermethylation or other epigenetic mechanisms including, but not limited to, IGF2 and SAT2.

With respect to TP53, the therapeutic mRNA may encode any of the corresponding wild type isoforms listed in the National Center for Biotechnology Information database for Gene ID 7157. A representative protein sequence from isoform “a” transcript variant “1” is listed below:

(SEQ ID NO: 84)
MEEPQSDPSVEPPLSQETFSDLWKLLPENNVLSPLPSQAMDDLMLSPDD
IEQWFTEDPGPDEAPRMPEAAPPVAPAPAAPTPAAPAPAPSWPLSSSVP
SQKTYQGSYGFRLGFLHSGTAKSVTCTYSPALNKMFCQLAKTCPVQLWV
DSTPPPGTRVRAMAIYKQSQHMTEVVRRCPHHERCSDSDGLAPPQHLIR
VEGNLRVEYLDDRNTFRHSVVVPYEPPEVGSDCTTIHYNYMCNSSCMGG
MNRRPILTIITLEDSSGNLLGRNSFEVRVCACPGRDRRTEEENLRKKGE
PHHELPPGSTKRALPNNTSSSPQPKKKPLDGEYFTLQIRGRERFEMFRE
LNEALELKDAQAGKEPGGSRAHSSHLKSKKGQSTSRHKKLMEKTEGPDS
D

Another wild type TP53 mRNA sequence for use as a therapeutic mRNA is found under Genbank accession number AF307851:

(SEQ ID NO: 85)
AUGGAGGAGCCGCAGUCAGAUCCUAGCGUCGAGCCCCCUCUGAGUCAGG
AAACAUUUUCAGACCUAUGGAAACUACUUCCUGAAAACAACGUUCUGUC
CCCCUUGCCGUCCCAAGCAAUGGAUGAUUUGAUGCUGUCCCCGGACGAU
AUUGAACAAUGGUUCACUGAAGACCCAGGUCCAGAUGAAGCUCCCAGAA
UGCCAGAGGCUGCUCCCCGCGUGGCCCCUGCACCAGCAGCUCCUACACC
GGCGGCCCCUGCACCAGCCCCCUCCUGGCCCCUGUCAUCUUCUGUCCCU
UCCCAGAAAACCUACCAGGGCAGCUACGGUUUCCGUCUGGGCUUCUUGC
AUUCUGGGACAGCCAAGUCUGUGACUUGCACGUACUCCCCUGCCCUCAA
CAAGAUGUUUUGCCAACUGGCCAAGACCUGCCCUGUGCAGCUGUGGGUU
GAUUCCACACCCCCGCCCGGCACCCGCGUCCGCGCCAUGGCCAUCUACA
AGCAGUCACAGCACAUGACGGAGGUUGUGAGGCGCUGCCCCCACCAUGA
GCGCUGCUCAGAUAGCGAUGGUCUGGCCCCUCCUCAGCAUCUUAUCCGA
GUGGAAGGAAAUUUGCGUGUGGAGUAUUUGGAUGACAGAAACACUUUUC
GACAUAGUGUGGUGGUGCCCUAUGAGCCGCCUGAGGUUGGCUCUGACUG
UACCACCAUCCACUACAACUACAUGUGUAACAGUUCCUGCAUGGGCGGC
AUGAACCGGAGGCCCAUCCUCACCAUCAUCACACUGGAAGACUCCAGUG
GUAAUCUACUGGGACGGAACAGCUUUGAGGUGCGUGUUUGUGCCUGUCC
UGGGAGAGACCGGCGCACAGAGGAAGAGAAUCUCCGCAAGAAAGGGGAG
CCUCACCACGAGCUGCCCCCAGGGAGCACUAAGCGAGCACUGCCCAACA
ACACCAGCUCCUCUCCCCAGCCAAAGAAGAAACCACUGGAUGGAGAAUA
UUUCACCCUUCAGAUCCGUGGGCGUGAGCGCUUCGAGAUGUUCCGAGAG
CUGAAUGAGGCCUUGGAACUCAAGGAUGCCCAGGCUGGGAAGGAGCCAG
GGGGGAGCAGGGCUCACUCCAGCCACCUGAAGUCCAAAAAGGGUCAGUC
UACCUCCCGCCAUAAAAAACUCAUGUUCAAGACAGAAGGGCCUGACUCA
GACAAGGGCUCGAGUCUAGAGGGCCCGCGGUUCGAAGGUAAGCCUAUCC
CUAACCCUCUCCUCGGUCUCGAUUCUACGCGUACCGGUUAG

With respect to CRISPR/Cas endonucleases which may be incorporated in vectors (e.g., LNPs) in either ribonucleoprotein or mRNA forms, a representative protein sequence for high fidelity (HiFi) Cas9 endonuclease is listed below:

(SEQ ID NO: 82)
DKKYSIGLDIGTNSVGWAVITDEYKVPSKKFKVLGNTDRHSIKKNLIGA
LLFDSGETAEATRLKRTARRRYTRRKNRICYLQEIFSNEMAKVDDSFFH
RLEESFLVEEDKKHERHPIFGNIVDEVAYHEKYPTIYHLRKKLVDSTDK
ADLRLIYLALAHMIKFRGHFLIEGDLNPDNSDVDKLFIQLVQTYNQLFE
ENPINASGVDAKAILSARLSKSRRLENLIAQLPGEKKNGLFGNLIALSL
GLTPNFKSNFDLAEDAKLQLSKDTYDDDLDNLLAQIGDQYADLFLAAKN
LSDAILLSDILRVNTEITKAPLSASMIKRYDEHHQDLTLLKALVRQQLP
EKYKEIFFDQSKNGYAGYIDGGASQEEFYKFIKPILEKMDGTEELLVKL
NREDLLRKQRTFDNGSIPHQIHLGELHAILRRQEDFYPFLKDNREKIEK
ILTFRIPYYVGPLARGNSRFAWMTRKSEETITPWNFEEVVDKGASAQSF
IERMTNFDKNLPNEKVLPKHSLLYEYFTVYNELTKVKYVTEGMRKPAFL
SGEQKKAIVDLLFKTNRKVTVKQLKEDYFKKIECFDSVEISGVEDRFNA
SLGTYHDLLKIIKDKDFLDNEENEDILEDIVLTLTLFEDREMIEERLKT
YAHLFDDKVMKQLKRRRYTGWGRLSRKLINGIRDKQSGKTILDFLKSDG
FANANFMQLIHDDSLTFKEDIQKAQVSGQGDSLHEHIANLAGSPAIKKG
ILQTVKVVDELVKVMGRHKPENIVIEMARENQTTQKGQKNSRERMKRIE
EGIKELGSQILKEHPVENTQLQNEKLYLYYLQNGRDMYVDQELDINRLS
DYDVDHIVPQSFLKDDSIDNKVLTRSDKNRGKSDNVPSEEVVKKMKNYW
RQLLNAKLITQRKFDNLTKAERGGLSELDKAGFIKRQLVETRQITKHVA
QILDSRMNTKYDENDKLIREVKVITLKSKLVSDFRKDFQFYKVREINNY
HHAHDAYLNAVVGTALIKKYPKLESEFVYGDYKVYDVRKMIAKSEQEIG
KATAKYFFYSNIMNFFKTEITLANGEIRKRPLIETNGETGEIVWDKGRD
FATVRKVLSMPQVNIVKKTEVQTGGFSKESILPKRNSDKLIARKKDWDP
KKYGGFDSPTVAYSVLVVAKVEKGKSKKLKSVKELLGITIMERSSFEKN
PIDFLEAKGYKEVKKDLIIKLPKYSLFELENGRKRMLASAGELQKGNEL
ALPSKYVNFLYLASHYEKLKGSPEDNEQKQLFVEQHKHYLDEIIEQISE
FSKRVILADANLDKVLSAYNKHRDKPIREQAENIIHLFTLTNLGAPAAF
KYFDTTIDRKRYTSTKEVLDATLIHQSITGLYETRIDLSQLGGD

An additional Cas9 mRNA sequence that may be utilized is a modified mRNA sold by TriLink Biotechnologies (Catalog Number L-7206) provided below:

(SEQ IDNO: 83)
AUGGCCCCCAAGAAGAAGCGGAAGGUGGGCAUCCACGGCGUGCCCGCCG
CCGACAAGAAGUACAGCAUCGGCCUGGACAUCGGCACCAACAGCGUGGG
CUGGGCCGUGAUCACCGACGAGUACAAGGUGCCCAGCAAGAAGUUCAAG
GUGCUGGGCAACACCGACCGGCACAGCAUCAAGAAGAACCUGAUCGGCG
CCCUGCUGUUCGACAGCGGCGAGACCGCCGAGGCCACCCGGCUGAAGCG
GACCGCCCGGCGGCGGUACACCCGGCGGAAGAACCGGAUCUGCUACCUG
CAGGAGAUCUUCAGCAACGAGAUGGCCAAGGUGGACGACAGCUUCUUCC
ACCGGCUGGAGGAGAGCUUCCUGGUGGAGGAGGACAAGAAGCACGAGCG
GCACCCCAUCUUCGGCAACAUCGUGGACGAGGUGGCCUACCACGAGAAG
UACCCCACCAUCUACCACCUGCGGAAGAAGCUGGUGGACAGCACCGACA
AGGCCGACCUGCGGCUGAUCUACCUGGCCCUGGCCCACAUGAUCAAGUU
CCGGGGCCACUUCCUGAUCGAGGGCGACCUGAACCCCGACAACAGCGAC
GUGGACAAGCUGUUCAUCCAGCUGGUGCAGACCUACAACCAGCUGUUCG
AGGAGAACCCCAUCAACGCCAGCGGCGUGGACGCCAAGGCCAUCCUGAG
CGCCCGGCUGAGCAAGAGCCGGCGGCUGGAGAACCUGAUCGCCCAGCUG
CCCGGCGAGAAGAAGAACGGCCUGUUCGGCAACCUGAUCGCCCUGAGCC
UGGGCCUGACCCCCAACUUCAAGAGCAACUUCGACCUGGCCGAGGACGC
CAAGCUGCAGCUGAGCAAGGACACCUACGACGACGACCUGGACAACCUG
CUGGCCCAGAUCGGCGACCAGUACGCCGACCUGUUCCUGGCCGCCAAGA
ACCUGAGCGACGCCAUCCUGCUGAGCGACAUCCUGCGGGUGAACACCGA
GAUCACCAAGGCCCCCCUGAGCGCCAGCAUGAUCAAGCGGUACGACGAG
CACCACCAGGACCUGACCCUGCUGAAGGCCCUGGUGCGGCAGCAGCUGC
CCGAGAAGUACAAGGAGAUCUUCUUCGACCAGAGCAAGAACGGCUACGC
CGGCUACAUCGACGGCGGCGCCAGCCAGGAGGAGUUCUACAAGUUCAUC
AAGCCCAUCCUGGAGAAGAUGGACGGCACCGAGGAGCUGCUGGUGAAGC
UGAACCGGGAGGACCUGCUGCGGAAGCAGCGGACCUUCGACAACGGCAG
CAUCCCCCACCAGAUCCACCUGGGCGAGCUGCACGCCAUCCUGCGGCGG
CAGGAGGACUUCUACCCCUUCCUGAAGGACAACCGGGAGAAGAUCGAGA
AGAUCCUGACCUUCCGGAUCCCCUACUACGUGGGCCCCCUGGCCCGGGG
CAACAGCCGGUUCGCCUGGAUGACCCGGAAGAGCGAGGAGACCAUCACC
CCCUGGAACUUCGAGGAGGUGGUGGACAAGGGCGCCAGCGCCCAGAGCU
UCAUCGAGCGGAUGACCAACUUCGACAAGAACCUGCCCAACGAGAAGGU
GCUGCCCAAGCACAGCCUGCUGUACGAGUACUUCACCGUGUACAACGAG
CUGACCAAGGUGAAGUACGUGACCGAGGGCAUGCGGAAGCCCGCCUUCC
UGAGCGGCGAGCAGAAGAAGGCCAUCGUGGACCUGCUGUUCAAGACCAA
CCGGAAGGUGACCGUGAAGCAGCUGAAGGAGGACUACUUCAAGAAGAUC
GAGUGCUUCGACAGCGUGGAGAUCAGCGGCGUGGAGGACCGGUUCAACG
CCAGCCUGGGCACCUACCACGACCUGCUGAAGAUCAUCAAGGACAAGGA
CUUCCUGGACAACGAGGAGAACGAGGACAUCCUGGAGGACAUCGUGCUG
ACCCUGACCCUGUUCGAGGACCGGGAGAUGAUCGAGGAGCGGCUGAAGA
CCUACGCCCACCUGUUCGACGACAAGGUGAUGAAGCAGCUGAAGCGGCG
GCGGUACACCGGCUGGGGCCGGCUGAGCCGGAAGCUGAUCAACGGCAUC
CGGGACAAGCAGAGCGGCAAGACCAUCCUGGACUUCCUGAAGAGCGACG
GCUUCGCCAACCGGAACUUCAUGCAGCUGAUCCACGACGACAGCCUGAC
CUUCAAGGAGGACAUCCAGAAGGCCCAGGUGAGCGGCCAGGGCGACAGC
CUGCACGAGCACAUCGCCAACCUGGCCGGCAGCCCCGCCAUCAAGAAGG
GCAUCCUGCAGACCGUGAAGGUGGUGGACGAGCUGGUGAAGGUGAUGGG
CCGGCACAAGCCCGAGAACAUCGUGAUCGAGAUGGCCCGGGAGAACCAG
ACCACCCAGAAGGGCCAGAAGAACAGCCGGGAGCGGAUGAAGCGGAUCG
AGGAGGGCAUCAAGGAGCUGGGCAGCCAGAUCCUGAAGGAGCACCCCGU
GGAGAACACCCAGCUGCAGAACGAGAAGCUGUACCUGUACUACCUGCAG
AACGGCCGGGACAUGUACGUGGACCAGGAGCUGGACAUCAACCGGCUGA
GCGACUACGACGUGGACCACAUCGUGCCCCAGAGCUUCCUGAAGGACGA
CAGCAUCGACAACAAGGUGCUGACCCGGAGCGACAAGAACCGGGGCAAG
AGCGACAACGUGCCCAGCGAGGAGGUGGUGAAGAAGAUGAAGAACUACU
GGCGGCAGCUGCUGAACGCCAAGCUGAUCACCCAGCGGAAGUUCGACAA
CCUGACCAAGGCCGAGCGGGGCGGCCUGAGCGAGCUGGACAAGGCCGGC
UUCAUCAAGCGGCAGCUGGUGGAGACCCGGCAGAUCACCAAGCACGUGG
CCCAGAUCCUGGACAGCCGGAUGAACACCAAGUACGACGAGAACGACAA
GCUGAUCCGGGAGGUGAAGGUGAUCACCCUGAAGAGCAAGCUGGUGAGC
GACUUCCGGAAGGACUUCCAGUUCUACAAGGUGCGGGAGAUCAACAACU
ACCACCACGCCCACGACGCCUACCUGAACGCCGUGGUGGGCACCGCCCU
GAUCAAGAAGUACCCCAAGCUGGAGAGCGAGUUCGUGUACGGCGACUAC
AAGGUGUACGACGUGCGGAAGAUGAUCGCCAAGAGCGAGCAGGAGAUCG
GCAAGGCCACCGCCAAGUACUUCUUCUACAGCAACAUCAUGAACUUCUU
CAAGACCGAGAUCACCCUGGCCAACGGCGAGAUCCGGAAGCGGCCCCUG
AUCGAGACCAACGGCGAGACCGGCGAGAUCGUGUGGGACAAGGGCCGGG
ACUUCGCCACCGUGCGGAAGGUGCUGAGCAUGCCCCAGGUGAACAUCGU
GAAGAAGACCGAGGUGCAGACCGGCGGCUUCAGCAAGGAGAGCAUCCUG
CCCAAGCGGAACAGCGACAAGCUGAUCGCCCGGAAGAAGGACUGGGACC
CCAAGAAGUACGGCGGCUUCGACAGCCCCACCGUGGCCUACAGCGUGCU
GGUGGUGGCCAAGGUGGAGAAGGGCAAGAGCAAGAAGCUGAAGAGCGUG
AAGGAGCUGCUGGGCAUCACCAUCAUGGAGCGGAGCAGCUUCGAGAAGA
ACCCCAUCGACUUCCUGGAGGCCAAGGGCUACAAGGAGGUGAAGAAGGA
CCUGAUCAUCAAGCUGCCCAAGUACAGCCUGUUCGAGCUGGAGAACGGC
CGGAAGCGGAUGCUGGCCAGCGCCGGCGAGCUGCAGAAGGGCAACGAGC
UGGCCCUGCCCAGCAAGUACGUGAACUUCCUGUACCUGGCCAGCCACUA
CGAGAAGCUGAAGGGCAGCCCCGAGGACAACGAGCAGAAGCAGCUGUUC
GUGGAGCAGCACAAGCACUACCUGGACGAGAUCAUCGAGCAGAUCAGCG
AGUUCAGCAAGCGGGUGAUCCUGGCCGACGCCAACCUGGACAAGGUGCU
GAGCGCCUACAACAAGCACCGGGACAAGCCCAUCCGGGAGCAGGCCGAG
AACAUCAUCCACCUGUUCACCCUGACCAACCUGGGCGCCCCCGCCGCCU
UCAAGUACUUCGACACCACCAUCGACCGGAAGCGGUACACCAGCACCAA
GGAGGUGCUGGACGCCACCCUGAUCCACCAGAGCAUCACCGGCCUGUAC
GAGACCCGGAUCGACCUGAGCCAGCUGGGCGGCGACAGCGGCGGCAAGC
GGCCCGCCGCCACCAAGAAGGCCGGCCAGGCCAAGAAGAAGAAGGGCAG
CUACCCCUACGACGUGCCCGACUACGCCUGA

The donor therapeutic mRNAs and Cas9 mRNA sequences may incorporate features that contribute to mRNA stability by preventing premature decapping and resisting exoribonuclease activity which include but are not limited to utilizing codon optimization, a synthetic 5′ UTR with a strong consensus kozak and a mouse alpha-globin 3′ UTR, optimized for high expression within mammalian cell lines with mRNA capping such as with CleanCap (Cap1) technology (TriLink), and adding a 120A tail. Other mRNA expression methods to enhance expression are known and may be utilized.

### Targeted Lipid Nanoparticles (LNP)

In certain embodiments, the vector for delivering the components for synergic editing may be a lipid nanoparticle (LNP). In some aspects, the components of LNPs may include positively charged ionizable cationic lipids, neutral ionizable lipids, phospholipids, cholesterol, and polyethylene glycol (PEG)-lipids. In particular embodiments, LNPs contain a helper lipid and/or targeting moieties to promote cell binding, cholesterol to fill the gaps between the lipids, and a polyethylene glycol (PEG) to reduce opsonization by serum proteins and reticuloendothelial clearance. LNP preparations with an antibody targeting moiety may be synthesized according to known methods and may be utilized to deliver the components described above for synergic editing donor therapeutic DNA and mRNA vectors. In certain embodiments, LNP components may include DLin-MC3-DMA (MC3) Cholesterol, DSPC (1,2-distearoyl-sn-glycero-3-phosphocholine), polyethylene glycol (PEG)-DMG (1,2-dimyristoyl-rac-glycerol), and DSPE (1,2-distearoyl-sn-glycero-3-phosphoethanolamine)-PEG, which are commercially available from suppliers such as Avanti Polar Lipids Inc. To incorporate the antibody targeting moieties into the LNPs, lipidated antibody components may be incubated with the LNPs using known methods. In certain cancer treatment embodiments described herein, anti-human EGFR antibody or anti-human CD38 antibodies may be employed (available from suppliers such as Bio-Rad Laboratories Inc or Bio X Cell, NH, USA).

LNP preparations with a CD38 targeting moiety may be synthesized according to known methods. The lipids used for CD38-targeted LNP production (Cholesterol, DSPC and DSPE PEG-Malemide) may be purchased from Avanti Polar lipids (USA). Dlin-MC3-DMA may be synthesized according to known methods. CD38-targeted LNPs may be prepared using microfluidic micro mixture (Precision NanoSystems, Vancouver, BC, Canada) by known methods comprising Dlin-MC3-DMA, DSPC, Chol, DMG-PEG and DSPE-PEG Mal. In certain aspects, an anti-CD38 monoclonal Ab (clone THB-7) may be reduced to allow its chemical conjugation to maleimide groups present in the LNPs and then incubated with the LNPs.

Table 5 provides a representative and non-limiting list of targeting antibodies that may be utilized for synergic editing with LNP vectors for cancer treatment.

TABLE 5
Antibodies to Target Synergic Editing LNP
Vectors for Cancer Treatment

| Condition | Target |
|---|---|
| Ovarian, Colorectal, Head and | EGFR |
| Neck and other EGFR positive | |
| cancers | |
| Mantle Cell Lymphoma (MCL) | CD38 |
| and other CD38 positive cancers | |
| B cell lymphomas and other CD20 | CD20 |
| positive cancers | |
| Neuroblastoma and other GD2 | GD2 |
| positive cancers | |
| Acute myelogenous leukemia | CD33 |
| (AML) and other CD33 positive | |
| cancers | |
| Hodgkin Lymphoma (HL) and | CD30 |
| Anaplastic large cell lymphoma | |
| (ALCL) and other and other CD30 | |
| positive cancers | |
| HER2-positive breast cancer and | HER2 |
| other HER2-positive cancers | |
| CD22-positive B-cell precursor | CD22 |
| acute lymphoblastic leukemia, | |
| hairy cell leukemia (HCL) and | |
| other CD22 positive cancers | |
| Diffuse large B-cell lymphoma | CD79 |
| (DLBCL) and other CD79 positive | |
| cancers | |
| Urothelial cancer and other | Nectin-4 |
| Nectin-4 positive cancers | |
| Triple-negative breast cancer | Trop-2 |
| (mTNBC) and other Trop-2 | |
| positive cancers | |
| Multiple myeloma and other | BCMA |
| BCMA positive cancers | |
| Large B-cell lymphoma and other | CD19 |
| CD19 positive cancers | |
| Cervical cancer and other Tissue | Tissue factor |
| Factor positive cancers | |

### Viral Vector Delivery

In addition or alternatively to LNPs, in certain embodiments, viral vectors may be employed to deliver synergic editing components, such as those described above and shown in FIGS. 2 and 4. In some embodiments, the viral vector is an adenovirus, an adeno-associated virus, a lentivirus, a herpes virus, or any other viral vector used for other forms of gene editing and gene therapy.

### Definitions

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

The terms “about”, “substantially” and “approximately” mean, in general, the stated value plus or minus 5%.

For Synergic Editing with Donor Therapeutic mRNA Vectors which employ donor therapeutic mRNA for synergic editing, the syn-gRNAs are conventional single guide RNAs which complex with an endonuclease to disrupt pathogenic genes of interest in the host genome. For Synergic Editing with Donor Therapeutic mRNA Vectors, the terms syn-gRNA, single guide RNA, and sgRNA are equivalent.

The term “exogenous,” when used in relation to a protein, gene, nucleic acid, or polynucleotide in a cell or organism refers to a protein, gene, nucleic acid, or polynucleotide that has been introduced into the cell or organism by artificial or natural means; or in relation to a cell, the term refers to a cell that was isolated and subsequently introduced to other cells or to an organism by artificial or natural means. An exogenous nucleic acid may be from a different organism or cell, or it may be one or more additional copies of a nucleic acid that occurs naturally within the organism or cell. An exogenous cell may be from a different organism, or it may be from the same organism. By way of a non-limiting example, an exogenous nucleic acid is one that is in a chromosomal location different from where it would be in natural cells, or is otherwise flanked by a different nucleic acid sequence than that found in nature.

By “expression construct” or “expression cassette” is meant a nucleic acid molecule that is capable of directing transcription. An expression construct includes, at a minimum, one or more transcriptional control elements (such as promoters, enhancers or a structure functionally equivalent thereof) that direct gene expression in one or more desired cell types, tissues or organs. Additional elements, such as a transcription termination signal, may also be included.

A “vector” or “construct” (sometimes referred to as a gene delivery system or gene transfer “vehicle”) refers to a macromolecule or complex of molecules comprising a polynucleotide to be delivered to a host cell, either in vitro or in vivo.

A “plasmid,” a common type of a vector, is an extra-chromosomal DNA molecule separate from the chromosomal DNA that is capable of replicating independently of the chromosomal DNA. In certain cases, it is circular and double-stranded.

“Homology” refers to the percent of identity between two polynucleotides or two polypeptides. The correspondence between one sequence and another can be determined by techniques known in the art. For example, homology can be determined by a direct comparison of the sequence information between two polypeptide molecules by aligning the sequence information and using readily available computer programs. Alternatively, homology can be determined by hybridization of polynucleotides under conditions that promote the formation of stable duplexes between homologous regions, followed by digestion with single strand-specific nuclease(s), and size determination of the digested fragments. Two DNA, or two polypeptide, sequences are “substantially homologous” to each other when at least about 80%, preferably at least about 90%, and most preferably at least about 95% of the nucleotides, or amino acids, respectively match over a defined length of the molecules, as determined using the methods above.

“Treating” or treatment of a disease or condition refers to executing a protocol, which may include administering one or more drugs to a patient, in an effort to alleviate signs or symptoms of the disease. Desirable effects of treatment include decreasing the rate of disease progression, ameliorating or palliating the disease state, and remission or improved prognosis. Alleviation can occur prior to signs or symptoms of the disease or condition appearing, as well as after their appearance. Thus, “treating” or “treatment” may include “preventing” or “prevention” of disease or undesirable condition. In addition, “treating” or “treatment” does not require complete alleviation of signs or symptoms, does not require a cure, and specifically includes protocols that have only a marginal effect on the patient.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the size of a tumor, a reduction in the invasiveness of a tumor, reduction in the growth rate of the cancer, or prevention of metastasis. Treatment of cancer may also refer to prolonging survival of a subject with cancer.

“Subject” and “patient” refer to either a human or non-human, such as primates, mammals, and vertebrates. In particular embodiments, the subject is a human.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds disclosed herein which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2ethanedisulfonic acid, 2hydroxyethanesulfonic acid, 2naphthalenesulfonic acid, 3phenylpropionic acid, 4,4′ methylenebis(3hydroxy2ene-1carboxylic acid), 4methylbicyclo[2.2.2]oct2ene-1carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o(4hydroxybenzoyl)benzoic acid, oxalic acid, pchlorobenzenesulfonic acid, phenyl-substituted alkanoic acids, propionic acid, ptoluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N methylglucamine and the like. It should be recognized that the particular anion or cation forming a part of any salt of this disclosure is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

A “pharmaceutically acceptable carrier,” “drug carrier,” or simply “carrier” is a pharmaceutically acceptable substance formulated along with the active ingredient medication that is involved in carrying, delivering and/or transporting a chemical agent. Drug carriers may be used to improve the delivery and the effectiveness of drugs, including for example, controlled-release technology to modulate drug bioavailability, decrease drug metabolism, and/or reduce drug toxicity. Some drug carriers may increase the effectiveness of drug delivery to the specific target sites. Examples of carriers include: lipid nanoparticles, liposomes, microspheres (e.g., made of poly(lactic-co-glycolic) acid), albumin microspheres, synthetic polymers, nanofibers, protein-DNA complexes, protein conjugates, erythrocytes, virosomes, and dendrimers.

### 2. Regulatory Elements

Expression cassettes included in vectors useful in the present disclosure in particular contain (in a 5′-to-3′ direction) a eukaryotic transcriptional promoter operably linked to a protein-coding sequence, splice signals including intervening sequences, and a transcriptional termination/polyadenylation sequence. The promoters and enhancers that control the transcription of protein encoding genes in eukaryotic cells are composed of multiple genetic elements. The cellular machinery is able to gather and integrate the regulatory information conveyed by each element, allowing different genes to evolve distinct, often complex patterns of transcriptional regulation. A promoter used in the context of the present disclosure includes constitutive, inducible, and tissue-specific promoters.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al. 1989, incorporated herein by reference). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Additionally, any promoter/enhancer combination (as per, for example, the Eukaryotic Promoter Data Base EPDB, through world wide web at epd.isb-sib.ch/) could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

Non-limiting examples of promoters include early or late viral promoters, such as, SV40 early or late promoters, cytomegalovirus (CMV) immediate early promoters, Rous Sarcoma Virus (RSV) early promoters; eukaryotic cell promoters, such as, e.g., beta actin promoter (Ng, 1989; Quitsche et al., 1989), GADPH promoter (Alexander et al., 1988, Ercolani et al., 1988), metallothionein promoter (Karin et al., 1989; Richards et al., 1984); and concatenated response element promoters, such as cyclic AMP response element promoters (cre), serum response element promoter (sre), phorbol ester promoter (TPA) and response element promoters (tre) near a minimal TATA box. It is also possible to use human growth hormone promoter sequences (e.g., the human growth hormone minimal promoter described at Genbank, accession no. X05244, nucleotide 283-341) or a mouse mammary tumor promoter (available from the ATCC, Cat. No. ATCC 45007). In certain embodiments, the promoter is CMV IE, dectin-1, dectin-2, human CD11c, F4/80, SM22, RSV, SV40, Ad MLP, beta-actin, MHC class I or MHC class II promoter, however any other promoter that is useful to drive expression of the p53, MDA-7 and/or the relaxin gene is applicable to the practice of the present disclosure.

In certain aspects, methods of the disclosure also concern enhancer sequences, i.e., nucleic acid sequences that increase a promoter's activity and that have the potential to act in cis, and regardless of their orientation, even over relatively long distances (up to several kilobases away from the target promoter). However, enhancer function is not necessarily restricted to such long distances as they may also function in close proximity to a given promoter.

b. Initiation Signals and Linked Expression

A specific initiation signal also may be used in the expression constructs provided in the present disclosure for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments, the use of internal ribosome entry sites (IRES) elements are used to create multigene, or polycistronic, messages. IRES elements are able to bypass the ribosome scanning model of 5′ methylated Cap dependent translation and begin translation at internal sites. IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

c. Origins of Replication

In order to propagate a vector in a host cell, it may contain one or more origins of replication sites (often termed “ori”), for example, a nucleic acid sequence corresponding to oriP of EBV as described above or a genetically engineered oriP with a similar or elevated function in programming, which is a specific nucleic acid sequence at which replication is initiated. Alternatively a replication origin of other extra-chromosomally replicating virus as described above or an autonomously replicating sequence (ARS) can be employed.

### 3. Selection and Screenable Markers

In some embodiments, cells containing a construct of the present disclosure may be identified in vitro or in vivo by including a marker in the expression vector. Such markers would confer an identifiable change to the cell permitting easy identification of cells containing the expression vector. Generally, a selection marker is one that confers a property that allows for selection. A positive selection marker is one in which the presence of the marker allows for its selection, while a negative selection marker is one in which its presence prevents its selection. An example of a positive selection marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selection markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes as negative selection markers such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selection and screenable markers are well known to one of skill in the art.

### EXAMPLES

The following examples are included to demonstrate specific embodiments of the disclosure. The present teachings include descriptions provided in the examples that are not intended to limit the scope of any aspect or claim. The following non-limiting examples are provided to further illustrate the present teachings. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Synergic Editing Demonstrates Superior Efficacy Compared to Standard Gene Editing In Vitro Results

In these in vitro experiments, donor therapeutic DNA and mRNA LNP vectors for synergic editing are constructed, and their superior efficacy for cancer treatment is demonstrated compared to standard gene editing LNP vectors using tumor cell lines from a variety of cancer types. Standard gene editing employs separate LNP vectors for gene disruptions and therapeutic nucleic acid expression. The specific tumor cell lines, LNP compositions, guide RNAs, Cas endonuclease, therapeutic mRNAs and donor therapeutic DNAs for these applications are described in detail below.

Tumor cell lines include OVCAR8, HepG2, HCT116 (ATCC, CRL-247), A549 (ATCC, CRL-185), and Granta-519 (purchased from DSMZ, Germany) maintained in DMEM or RPMI-1640 (Gibco, Thermo-Fisher Scientific, Inc) media supplemented with 10% FBS, L-glutamine and Penicillin-Streptomycin-Nystatin. In the examples of synergic editing involving donor therapeutic RNAs, cell lines representative of different types of tumors were utilized-OVCAR8 (ovarian cancer) and HepG2 (hepatocellular carcinoma).

### Lnp Preparations.

In the examples of synergic editing involving donor therapeutic RNAs, DLin-MC3-DMA (MC3) and Lipid 8 LNPs are synthesized according to a previously described method. Cholesterol, DSPC (1,2-distearoyl-sn-glycero-3-phosphocholine), polyethylene glycol (PEG)-DMG (1,2-dimyristoyl-rac-glycerol), and DSPE (1,2-distearoyl-sn-glycero-3-phosphoethanolamine)-PEG are purchased from Avanti Polar Lipids Inc. Briefly, one volume of lipid mixture (ionizable lipid, DSPC, cholesterol, DMG-PEG, and DSPE-PEG at 50:10.5:38:1.4:0.1 molar ratio) in ethanol and three volumes of combined donor therapeutic mRNA and mCas9/sgRNA (mCas9:sgRNA 3:1 weight ratio, 1:10 molar ratio RNA to ionizable lipid) in a citrate buffer are injected into a NanoAssemblr microfluidic mixing device (Precision Nanosystems Inc.) at a combined flow rate of 12 ml min-1. The formed LNPs are dialyzed twice against PBS (pH 7.4) for 16 hours to remove ethanol. As controls, separate donor therapeutic RNA LNPs and mRNA Cas9/sgRNA LNPs were similarly constructed.

LNP preparations with an EGFR targeting moiety are synthesized according to previously described methods. DLin-MC3-DMA (MC3) Cholesterol, DSPC (1,2-distearoyl-sn-glycero-3-phosphocholine), polyethylene glycol (PEG)-DMG (1,2-dimyristoyl-rac-glycerol), and DSPE (1,2-distearoyl-sn-glycero-3-phosphoethanolamine)-PEG are purchased from Avanti Polar Lipids Inc. Briefly, one volume of lipid mixture (ionizable lipid, DSPC, cholesterol, DMG-PEG, and DSPE-PEG at 50:10.5:38:1.4:0.1 molar ratio) in ethanol and three volumes of mCas9/sgRNA (mCas9:sgRNA 3:1 weight ratio, 1:10 molar ratio RNA to ionizable lipid) in a citrate buffer is injected into a NanoAssemblr microfluidic mixing device (Precision Nanosystems Inc.) at a combined flow rate-1 formed LNPs were dialyzed twice against PBS for 16 hours to remove ethanol. The LNP component in these studies contain an EGFR targeting moiety. To incorporate the EGFR targeting moiety into the LNPs, lipidated EGFR antibody components are incubated with the LNPs for 48 hours at 4° C., using previously described methods. Anti-human EGFR antibody (Bio-Rad Laboratories Inc., clone ICR10) or ra (Bio X Cell, NH, USA, clone 2A3) are used.

LNP preparations with an CD38 targeting moiety are synthesized according to a previously described method. The lipids used for CD38 targeted LNPs production (Cholesterol, DSPC and DSPE PEG-Mal) are purchased from Avanti Polar lipids (USA). Dlin-MC3-DMA is synthesized according to previously described methods. CD38 targeted LNPs are prepared using microfluidic micro mixture (Precision NanoSystems, Vancouver, BC, Canada). One volume of lipid mixtures (Dlin-MC3-DMA, DSPC, Chol, DMG-PEG and DSPE-PEG Mal at 50:10:38:1.5:0.5 mole ratio or Dlin-MC3-DMA, Chol, DSPC, DMG-PEG, DSPE-PEG-Mal at 50:38:10:1.95:0.05 molar ratio, 9.64 nM total lipid concentration) in ethanol and appropriate volumes of either donor therapeutic DNA or mRNA and corresponding syn-gRNAs, Usyn-gRNAs, Cas mRNA or RNP complexes of syn-gRNAs, Usyn-gRNAs and Cas9 protein containing buffer solutions are mixed by using dual syringe pump (Model S200, kD Scientific, Holliston, MA) to drive the solutions through the micro mixer at a combined flow rate of 2 mL/minute (0.5 mL/min for ethanol and 1.5 mL/min for aqueous buffer). The resultant mixture is dialyzed against PBS (pH 7.4) for 16 h to remove ethanol. The lipid mixture includes the following approximate lipid compositions: Dlin-MC3-DMA (50 mol %), cholesterol (38%), DSPC (10%), DMG-PEG (1.95%) and DSPE-PEG-Maleimide (0.05%). An anti-CD38 monoclonal Ab (clone THB-7) is reduced to allow its chemical conjugation to maleimide groups present in the LNPs and then incubated with the LNPs.

Nucleic Acid Sequences-syn-gRNAs/single guide RNAs and Usyn-gRNAs are designed as listed in Table 1 and are custom synthesized (modified) by either Integrated DNA Technologies or Synthego. Cas endonuclease with nuclear localization signal sequences (NLS) is provided as either a CleanCap Cas9 mRNA (modified) purchased from TriLink BioTechnologies Inc or complexed with syn-gRNAs and Usyn-gRNAs as a ribonucloprotein (RNP) purchased from Aldevron Inc. The Cas9-syn-gRNAs and Usyn-gRNAs RNP complexes are prepared by mixing solutions of Cas9 proteins and syn-gRNAs and Usyn-gRNAs in buffers at equal volumes by self-assembly at room temperature. The mole ratios of Cas9 protein to syn-gRNAs and Usyn-gRNAs used are varied from 1/1 to 1/10.

Therapeutic mRNA and DNA—At least one donor therapeutic mRNA or at least one donor therapeutic DNA template from Table 3 are also included in the synergic editing LNP vectors. The therapeutic mRNAs are custom manufactured as capped and polyadenylated mRNAs ready for translation by the ribosome either unmodified, codon optimized or modified with 5-methoxyuridine by TriLink BioTechnologies Inc. The donor therapeutic DNA template is custom manufactured as either a supercoiled DNA minicircle or linear plasmid which have neither bacterial origin of replication nor antibiotic resistance genes. Therapeutic DNAs are custom manufactured as minicircles from PlasmidFactory Inc or as linear plasmids from TriLink BioTechnologies Inc.

Treatment Efficacy following LNP transfection In Vitro—Cells are counted using trypan blue (Biological Industries), and 0.1×10⁶ cells are placed in tissue culture 12-well plates (Greiner Bio-One, Germany) with 1 ml of growing medium. In these studies, tumor cells are incubated with either a synergic editing LNP vector or an equivalent LNP concentration of a mixture of separate gene disrupting and gene expressing standard editing LNP vectors combined to contain the same synergic LNP vector components described above. Additional control preparations include PBS administration and LNP vectors that are empty or are missing one of the gene editing/therapy components such as the therapeutic donor DNA, therapeutic mRNA, sgRNA, or Cas9 protein or Cas9 mRNA. Cells are incubated with the treatments in standard culture conditions for 24 to 120 hours. Then, cells are washed three times, incubated in a fresh culture medium, and collected for flow cytometry (72 to 96 hours) or cell cycle assays (24 to 48 hours), as described below. The efficacy of the various treatments is assessed by cell viability evaluated by flow cytometry using APC Annexin V (BioLegend Inc., 640941) and DAPI as recommended by the manufacturer. Data from at least 2×104 cells are acquired using CytoFLEX and the CytExpert software (Beckman Coulter, USA). Analyses are done with FlowJo software. Cell viability evaluation is done using the XTT Cell Proliferation Kit according to the manufacturer's recommendation. Cell viability is then compared between the treatment groups. The synergic editing LNP vectors are shown to be statistically superior to a mixture of separate standard editing LNP vectors combined to contain the same synergic LNP vector components by Student T test or ANOVA.

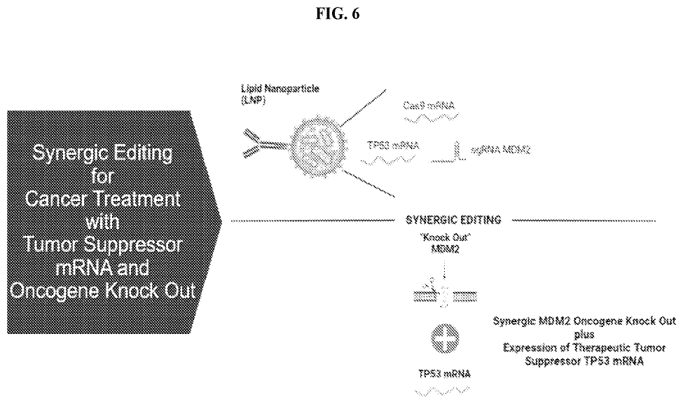

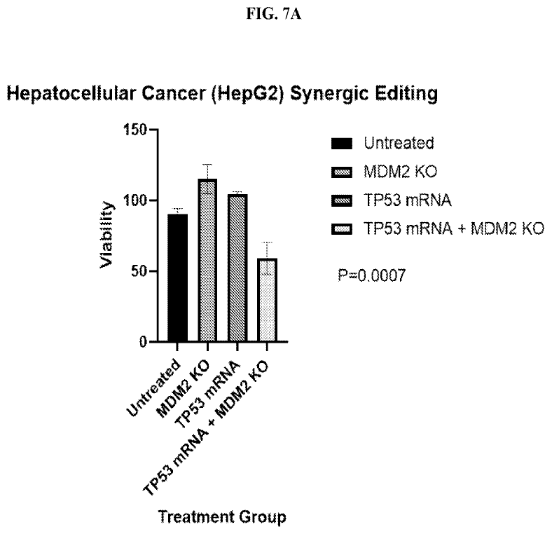

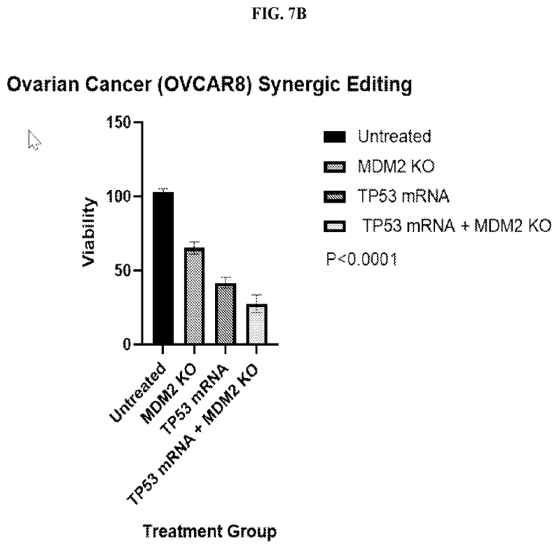

Exemplary results are shown in FIG. 6, FIG. 7A, and FIG. 7B of synergic editing involving donor therapeutic RNAs, a representative LNP vector comprising donor therapeutic RNA and a genome editing endonuclease mRNA and a single guide RNA guiding said genome editing endonuclease to cleave at least one pathogenic gene of interest resulting in the disruption of the pathogenic gene and expression of the donor therapeutic RNA sequence. As shown schematically in FIG. 6, the representative donor therapeutic mRNA encodes the tumor suppressor TP53 while the genome editing endonuclease mRNA is Cas9 and the guide RNA nucleic acid sequence knocks out the oncogene MDM2. The donor therapeutic mRNAs utilize a synthetic 5′ UTR with a strong consensus kozak and a mouse alpha-globin 3′ UTR, optimized for high expression within mammalian cell lines. Additionally, the mRNA is capped with CleanCap (Cap1) technology (TriLink), and a 120A tail is added. These features contribute to mRNA stability by preventing premature decapping and resisting exoribonuclease activity. For the experiments in this representative Example 1, synergic editing LNPs contain the donor therapeutic RNA encoding the tumor suppressor TP53, Cas9 mRNA and a guide RNA to the oncogene MDM2 as listed below.

The wild type TP53 therapeutic mRNA sequence utilized is under Genbank accession number AF307851:

(SEQ ID NO: 85)
AUGGAGGAGCCGCAGUCAGAUCCUAGCGUCGAGCCCCCUCUGAGUCAGG
AAACAUUUUCAGACCUAUGGAAACUACUUCCUGAAAACAACGUUCUGUC
CCCCUUGCCGUCCCAAGCAAUGGAUGAUUUGAUGCUGUCCCCGGACGAU
AUUGAACAAUGGUUCACUGAAGACCCAGGUCCAGAUGAAGCUCCCAGAA
UGCCAGAGGCUGCUCCCCGCGUGGCCCCUGCACCAGCAGCUCCUACACC
GGCGGCCCCUGCACCAGCCCCCUCCUGGCCCCUGUCAUCUUCUGUCCCU
UCCCAGAAAACCUACCAGGGCAGCUACGGUUUCCGUCUGGGCUUCUUGC
AUUCUGGGACAGCCAAGUCUGUGACUUGCACGUACUCCCCUGCCCUCAA
CAAGAUGUUUUGCCAACUGGCCAAGACCUGCCCUGUGCAGCUGUGGGUU
GAUUCCACACCCCCGCCCGGCACCCGCGUCCGCGCCAUGGCCAUCUACA
AGCAGUCACAGCACAUGACGGAGGUUGUGAGGCGCUGCCCCCACCAUGA
GCGCUGCUCAGAUAGCGAUGGUCUGGCCCCUCCUCAGCAUCUUAUCCGA
GUGGAAGGAAAUUUGCGUGUGGAGUAUUUGGAUGACAGAAACACUUUUC
GACAUAGUGUGGUGGUGCCCUAUGAGCCGCCUGAGGUUGGCUCUGACUG
UACCACCAUCCACUACAACUACAUGUGUAACAGUUCCUGCAUGGGCGGC
AUGAACCGGAGGCCCAUCCUCACCAUCAUCACACUGGAAGACUCCAGUG
GUAAUCUACUGGGACGGAACAGCUUUGAGGUGCGUGUUUGUGCCUGUCC
UGGGAGAGACCGGCGCACAGAGGAAGAGAAUCUCCGCAAGAAAGGGGAG
CCUCACCACGAGCUGCCCCCAGGGAGCACUAAGCGAGCACUGCCCAACA
ACACCAGCUCCUCUCCCCAGCCAAAGAAGAAACCACUGGAUGGAGAAUA
UUUCACCCUUCAGAUCCGUGGGCGUGAGCGCUUCGAGAUGUUCCGAGAG
CUGAAUGAGGCCUUGGAACUCAAGGAUGCCCAGGCUGGGAAGGAGCCAG
GGGGGAGCAGGGCUCACUCCAGCCACCUGAAGUCCAAAAAGGGUCAGUC
UACCUCCCGCCAUAAAAAACUCAUGUUCAAGACAGAAGGGCCUGACUCA
GACAAGGGCUCGAGUCUAGAGGGCCCGCGGUUCGAAGGUAAGCCUAUCC
CUAACCCUCUCCUCGGUCUCGAUUCUACGCGUACCGGUUAG

The Cas9 mRNA sequence employed is a modified mRNA sold by TriLink Biotechnologies (Catalog Number L-7206):

(SEQ ID NO: 83)
AUGGCCCCCAAGAAGAAGCGGAAGGUGGGCAUCCACGGCGUGCCCGCCG
CCGACAAGAAGUACAGCAUCGGCCUGGACAUCGGCACCAACAGCGUGGG
CUGGGCCGUGAUCACCGACGAGUACAAGGUGCCCAGCAAGAAGUUCAAG
GUGCUGGGCAACACCGACCGGCACAGCAUCAAGAAGAACCUGAUCGGCG
CCCUGCUGUUCGACAGCGGCGAGACCGCCGAGGCCACCCGGCUGAAGCG
GACCGCCCGGCGGCGGUACACCCGGCGGAAGAACCGGAUCUGCUACCUG
CAGGAGAUCUUCAGCAACGAGAUGGCCAAGGUGGACGACAGCUUCUUCC
ACCGGCUGGAGGAGAGCUUCCUGGUGGAGGAGGACAAGAAGCACGAGCG
GCACCCCAUCUUCGGCAACAUCGUGGACGAGGUGGCCUACCACGAGAAG
UACCCCACCAUCUACCACCUGCGGAAGAAGCUGGUGGACAGCACCGACA
AGGCCGACCUGCGGCUGAUCUACCUGGCCCUGGCCCACAUGAUCAAGUU
CCGGGGCCACUUCCUGAUCGAGGGCGACCUGAACCCCGACAACAGCGAC
GUGGACAAGCUGUUCAUCCAGCUGGUGCAGACCUACAACCAGCUGUUCG
AGGAGAACCCCAUCAACGCCAGCGGCGUGGACGCCAAGGCCAUCCUGAG
CGCCCGGCUGAGCAAGAGCCGGCGGCUGGAGAACCUGAUCGCCCAGCUG
CCCGGCGAGAAGAAGAACGGCCUGUUCGGCAACCUGAUCGCCCUGAGCC
UGGGCCUGACCCCCAACUUCAAGAGCAACUUCGACCUGGCCGAGGACGC
CAAGCUGCAGCUGAGCAAGGACACCUACGACGACGACCUGGACAACCUG
CUGGCCCAGAUCGGCGACCAGUACGCCGACCUGUUCCUGGCCGCCAAGA
ACCUGAGCGACGCCAUCCUGCUGAGCGACAUCCUGCGGGUGAACACCGA
GAUCACCAAGGCCCCCCUGAGCGCCAGCAUGAUCAAGCGGUACGACGAG
CACCACCAGGACCUGACCCUGCUGAAGGCCCUGGUGCGGCAGCAGCUGC
CCGAGAAGUACAAGGAGAUCUUCUUCGACCAGAGCAAGAACGGCUACGC
CGGCUACAUCGACGGCGGCGCCAGCCAGGAGGAGUUCUACAAGUUCAUC
AAGCCCAUCCUGGAGAAGAUGGACGGCACCGAGGAGCUGCUGGUGAAGC
UGAACCGGGAGGACCUGCUGCGGAAGCAGCGGACCUUCGACAACGGCAG
CAUCCCCCACCAGAUCCACCUGGGCGAGCUGCACGCCAUCCUGCGGCGG
CAGGAGGACUUCUACCCCUUCCUGAAGGACAACCGGGAGAAGAUCGAGA
AGAUCCUGACCUUCCGGAUCCCCUACUACGUGGGCCCCCUGGCCCGGGG
CAACAGCCGGUUCGCCUGGAUGACCCGGAAGAGCGAGGAGACCAUCACC
CCCUGGAACUUCGAGGAGGUGGUGGACAAGGGCGCCAGCGCCCAGAGCU
UCAUCGAGCGGAUGACCAACUUCGACAAGAACCUGCCCAACGAGAAGGU
GCUGCCCAAGCACAGCCUGCUGUACGAGUACUUCACCGUGUACAACGAG
CUGACCAAGGUGAAGUACGUGACCGAGGGCAUGCGGAAGCCCGCCUUCC
UGAGCGGCGAGCAGAAGAAGGCCAUCGUGGACCUGCUGUUCAAGACCAA
CCGGAAGGUGACCGUGAAGCAGCUGAAGGAGGACUACUUCAAGAAGAUC
GAGUGCUUCGACAGCGUGGAGAUCAGCGGCGUGGAGGACCGGUUCAACG
CCAGCCUGGGCACCUACCACGACCUGCUGAAGAUCAUCAAGGACAAGGA
CUUCCUGGACAACGAGGAGAACGAGGACAUCCUGGAGGACAUCGUGCUG
ACCCUGACCCUGUUCGAGGACCGGGAGAUGAUCGAGGAGCGGCUGAAGA
CCUACGCCCACCUGUUCGACGACAAGGUGAUGAAGCAGCUGAAGCGGCG
GCGGUACACCGGCUGGGGCCGGCUGAGCCGGAAGCUGAUCAACGGCAUC
CGGGACAAGCAGAGCGGCAAGACCAUCCUGGACUUCCUGAAGAGCGACG
GCUUCGCCAACCGGAACUUCAUGCAGCUGAUCCACGACGACAGCCUGAC
CUUCAAGGAGGACAUCCAGAAGGCCCAGGUGAGCGGCCAGGGCGACAGC
CUGCACGAGCACAUCGCCAACCUGGCCGGCAGCCCCGCCAUCAAGAAGG
GCAUCCUGCAGACCGUGAAGGUGGUGGACGAGCUGGUGAAGGUGAUGGG
CCGGCACAAGCCCGAGAACAUCGUGAUCGAGAUGGCCCGGGAGAACCAG
ACCACCCAGAAGGGCCAGAAGAACAGCCGGGAGCGGAUGAAGCGGAUCG
AGGAGGGCAUCAAGGAGCUGGGCAGCCAGAUCCUGAAGGAGCACCCCGU
GGAGAACACCCAGCUGCAGAACGAGAAGCUGUACCUGUACUACCUGCAG
AACGGCCGGGACAUGUACGUGGACCAGGAGCUGGACAUCAACCGGCUGA
GCGACUACGACGUGGACCACAUCGUGCCCCAGAGCUUCCUGAAGGACGA
CAGCAUCGACAACAAGGUGCUGACCCGGAGCGACAAGAACCGGGGCAAG
AGCGACAACGUGCCCAGCGAGGAGGUGGUGAAGAAGAUGAAGAACUACU
GGCGGCAGCUGCUGAACGCCAAGCUGAUCACCCAGCGGAAGUUCGACAA
CCUGACCAAGGCCGAGCGGGGCGGCCUGAGCGAGCUGGACAAGGCCGGC
UUCAUCAAGCGGCAGCUGGUGGAGACCCGGCAGAUCACCAAGCACGUGG
CCCAGAUCCUGGACAGCCGGAUGAACACCAAGUACGACGAGAACGACAA
GCUGAUCCGGGAGGUGAAGGUGAUCACCCUGAAGAGCAAGCUGGUGAGC
GACUUCCGGAAGGACUUCCAGUUCUACAAGGUGCGGGAGAUCAACAACU
ACCACCACGCCCACGACGCCUACCUGAACGCCGUGGUGGGCACCGCCCU
GAUCAAGAAGUACCCCAAGCUGGAGAGCGAGUUCGUGUACGGCGACUAC
AAGGUGUACGACGUGCGGAAGAUGAUCGCCAAGAGCGAGCAGGAGAUCG
GCAAGGCCACCGCCAAGUACUUCUUCUACAGCAACAUCAUGAACUUCUU
CAAGACCGAGAUCACCCUGGCCAACGGCGAGAUCCGGAAGCGGCCCCUG
AUCGAGACCAACGGCGAGACCGGCGAGAUCGUGUGGGACAAGGGCCGGG
ACUUCGCCACCGUGCGGAAGGUGCUGAGCAUGCCCCAGGUGAACAUCGU
GAAGAAGACCGAGGUGCAGACCGGCGGCUUCAGCAAGGAGAGCAUCCUG
CCCAAGCGGAACAGCGACAAGCUGAUCGCCCGGAAGAAGGACUGGGACC
CCAAGAAGUACGGCGGCUUCGACAGCCCCACCGUGGCCUACAGCGUGCU
GGUGGUGGCCAAGGUGGAGAAGGGCAAGAGCAAGAAGCUGAAGAGCGUG
AAGGAGCUGCUGGGCAUCACCAUCAUGGAGCGGAGCAGCUUCGAGAAGA
ACCCCAUCGACUUCCUGGAGGCCAAGGGCUACAAGGAGGUGAAGAAGGA
CCUGAUCAUCAAGCUGCCCAAGUACAGCCUGUUCGAGCUGGAGAACGGC
UGGCCCUGCCCAGCAAGUACGUGAACUUCCUGUACCUGGCCAGCCACUA
CGAGAAGCUGAAGGGCAGCCCCGAGGACAACGAGCAGAAGCAGCUGUUC
GUGGAGCAGCACAAGCACUACCUGGACGAGAUCAUCGAGCAGAUCAGCG
AGUUCAGCAAGCGGGUGAUCCUGGCCGACGCCAACCUGGACAAGGUGCU
GAGCGCCUACAACAAGCACCGGGACAAGCCCAUCCGGGAGCAGGCCGAG
AACAUCAUCCACCUGUUCACCCUGACCAACCUGGGCGCCCCCGCCGCCU
UCAAGUACUUCGACACCACCAUCGACCGGAAGCGGUACCGGAAGCGGAU
GCUGGCCAGCGCCGGCGAGCUGCAGAAGGGCAACGAGCACCAGCACCAA
GGAGGUGCUGGACGCCACCCUGAUCCACCAGAGCAUCACCGGCCUGUAC
GAGACCCGGAUCGACCUGAGCCAGCUGGGCGGCGACAGCGGCGGCAAGC
GGCCCGCCGCCACCAAGAAGGCCGGCCAGGCCAAGAAGAAGAAGGGCAG
CUACCCCUACGACGUGCCCGACUACGCCUGA

The MDM2 single guide RNA sequence utilized corresponds to position 68820351 positive strand is CTTGGTAGTAGTCAATCAGC (SEQ ID NO: 81) with PAM AGG obtained from Integrated DNA Technologies (IDT) Design ID: Hs.Cas9.MDM2.1.AA.

The following control and treatment LNPs (at equivalent doses of 0.25 μg/ml) are compared in separate experiments employing the representative cancers-Hepatocellular Carcinoma (HepG2) and Ovarian Cancer (OVCAR8): Untreated Control, MDM2 knockout LNP containing Cas9 mRNA+MDM2-sgRNA (MDM2 KO); LNP containing TP53 mRNA (TP53 mRNA) and the “Synergic” LNP containing TP53 mRNA+Cas9 mRNA+MDM2-sgRNA with sgMDM2 RNA and P53 mRNA 1:3 in one LNP (TP53 mRNA+MDM2 KO). The cell lines were plated at 1×10⁵ cells/well, transfected by EA405 with the different treatment LNPs and analyzed for cell viability by XTT assay at 96 hours by previously described methods.

The unexpected results are shown in FIG. 7A and FIG. 7B. In HepG2 hepatocellular carcinoma, treatment with either TP53 mRNA or MDM2 KO LNPs alone had no therapeutic effects with basically unchanged viability compared to the untreated control. Surprisingly, the TP53 mRNA+MDM2 KO LNP demonstrated unexpected synergic effects resulting in a greater than 40% decrease in viability which was statistically significant overall by ANOVA P=0.007. In addition, there was a statistically significant decrease in viability for the “synergic” TP53 mRNA+MDM2 KO LNP compared to either the TP53 mRNA LNP alone (P=0.0013) or the MDM2 KO LNP alone (P=0.0006). Similar unexpected and corroborating results are observed for the treatment of ovarian carcinoma. As shown in FIG. 7B for OVCAR8 ovarian carcinoma, the greatest decrease in viability (greater than 70%) was observed for the TP53 mRNA+MDM2 KO LNP group which demonstrated more than additive effects compared to the other groups with statistically confirmed synergy by ANOVA P<0.0001. Consistent with the results for hepatocellular carcinoma above, the ovarian carcinoma treatment also demonstrated a statistically significant decrease in viability for the “synergic” TP53 mRNA+MDM2 KO LNP compared to either the TP53 mRNA LNP alone (P=0.0205) or the MDM2 KO LNP alone (P=0.0001). Overall, these treatment findings demonstrate the unexpected, substantial and statistically significant synergy of the TP53 mRNA+MDM2 KO LNP compared to either the TP53 mRNA LNP alone or the MDM2 KO LNP alone. The observation of similar synergistic effects in the treatment of different tumor types documents the efficacy of synergic editing for cancer treatment with donor therapeutic mRNA and CRISPR/Cas knockout of pathological cancer genes in a single LNP delivery vector.

### Example 2—Synergic Editing Demonstrates Superior Efficacy Compared to Standard Gene Editing In Vivo Results

Solid Tumor Carcinoma Treatment. For in vivo studies representative of solid tumor carcinomas, eight-week-old female Hsd: Athymic Nude-Foxnlnu mice are injected with 3×106 OV8-mCherry cells intra-peritoneally. Fluorescence imaging (IVIS SpectrumCT, PerkinElmer Inc.) is performed weekly after tumor cell implantation to monitor tumor growth. Fluorescence analysis is performed using the Living Image software (PerkinElmer Inc.). OV8-bearing mice are injected intraperitoneally with EGFR-targeted synergic editing LNPs as described for the in vitro studies (0.75 mg/kg) containing syn-gRNAs and Usyn-gRNAs, a Cas9 endonuclease complexed with the syn-gRNAs and Usyn-gRNAs as a ribonucloprotein or as a CleanCap Cas9 mRNA (modified) purchased from TriLink BioTechnologies Inc; at least one therapeutic mRNA or at least one therapeutic DNA sequence from Table 3. In these studies, 10 and 17 days after tumor inoculation, intra-peritoneal treatment with a synergic editing LNP vector (0.75 mg/kg) is compared to an equivalent LNP concentration (0.75 mg/kg) of a mixture of separate standard editing gene disrupting and therapeutic nucleic acid expressing LNP vectors combined to contain the same synergic editing LNP vector components described above. Additional control preparations include PBS administration and LNP vectors that are empty or are missing one of the gene editing/therapy components such as the donor therapeutic DNA, therapeutic mRNA, sgRNA, or Cas9 protein or Cas9 mRNA. Tumor growth is monitored using mCherry fluorescence of OV8-mCherry cells by the IVIS in vivo imaging system. Data are presented in total flux (p/s)±SEM; n=10 per group. One-way ANOVA is used to demonstrate the statistically significant decrease in tumor growth for the synergic editing LNP treatment groups. In addition, statistically significant increased survival for the synergic editing LNP treatment groups is demonstrated by Kaplan-Meier curves and log rank test.

Hematopoietic Cancer Treatment. To further demonstrate the broad applicability of the disclosure, in vivo experiments are also performed in a human mantle cell lymphoma (MCL) model representative of hematopoietic cancers using CD38 targeted synergic editing and standard editing LNPs. For modeling MCL in vivo, Female C.B-17/IcrHsd-Prkdcscid mice are utilized. 2.5×10⁶ Granta-519 or Granta-GFP cells are intravenously injected into 8 weeks old mice. Mice are monitored daily and killed when disease symptoms appeared (15% reduction in body weight or hind leg paralysis). The therapeutic effect of synergic editing and standard editing LNPs on the survival of MCL-bearing mice are tested. Mice (n=10/group) are treated biweekly with 9 i.v. injections starting 5 days after tumor inoculation. Additional control preparations include PBS administration and LNP vectors that are empty or are missing one of the gene editing/therapy components such as the donor therapeutic DNA, therapeutic mRNA, sgRNA, or Cas9 protein or Cas9 mRNA. Statistically significant increased survival for the synergic editing LNP treatment groups is demonstrated by Kaplan-Meier curves and log rank test.

Summary: The cancer models described in the Examples use highly aggressive forms of cancer, known to be generally resistant to conventional therapies. The therapeutic efficacy of synergic editing is significantly superior compared to standard gene editing or standard mRNA therapy. The net effect of synergic expression of therapeutic nucleic acids with concurrent disruption of pathogenic genes provides an unprecedented ability to address multiple molecular abnormalities not previously achievable in the treatment of multigenic diseases. Representative treatment findings demonstrated the unexpected, substantial and statistically significant synergy of an LNP delivering therapeutic mRNA and CRISPR/Cas oncogene knockout components compared to either the therapeutic mRNA LNP alone or the oncogene knockout LNP alone. The observation of similar synergistic effects in the treatment of different tumor types documents the efficacy of synergic editing for cancer treatment with donor therapeutic mRNA and CRISPR/Cas knockout of pathological cancer genes in a single LNP delivery vector.

All the methods disclosed and claimed herein can be made and executed without undue experimentation considering the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

1. A vector comprising:
(a) at least one donor therapeutic mRNA comprising a nucleic acid sequence encoding at least one therapeutic moiety;
(b) a genome editing endonuclease or a nucleic acid sequence encoding a genome editing endonuclease; and
(c) one or more guide RNA (gRNA) sequences for guiding the genome editing endonuclease to cleave at least one pathogenic gene of interest, thereby resulting in the disruption of the pathogenic gene of interest and expression of the at least one therapeutic moiety.

2. The vector of claim 1, further comprising a targeting moiety.

3. The vector of claim 2, wherein the targeting moiety is a cancer cell targeting moiety.

4. The vector of claim 2 or 3, wherein the targeting moiety is selected from the group consisting of: an antibody, an antibody fragment, a nanobody, a receptor ligand, and any combination thereof.

5. The vector of claim 4, wherein the targeting moiety is configured to bind to a target and deliver the vector to cancer cells, pre-cancerous cells, or pathogenic cells in a tumor microenvironment.

6. The vector of claim 5, wherein the targeting moiety binds to a target selected from the group consisting of: EGFR, HER2, CD19, CD20, CD22, CD33, CD38, BCMA, Nectin-4, Trop-2, tissue factor, GD2, any target listed in Table 5, and any combination thereof.

7. The vector of claim 1, wherein the nucleic acid sequence encoding the at least one therapeutic moiety is selected from the group consisting of: a tumor suppressor gene, a pro-apoptotic gene, an immune stimulatory gene, a suicide gene, an anti-cancer gene silenced by hypermethylation or other epigenetic mechanisms, a secreted decoy receptor gene, and any combination thereof.

8. The vector of claim 7, wherein the at least one therapeutic moiety is selected from the group consisting of: p53, PTEN, Rb, IL24, APC, BAX, BAK, BCLX, an interleukin, an interferon, a CD122/132 agonist, a chemokine, HSP 70/90, Calreticulin, HMGB1, a Toll Like Receptor, CGAS, STING1, ARHI, RASSF1A, DLEC1, SPARC, DAB2, PLAGL1, RPS6KA2, PTEN, OPCML, BRCA2, ARL11, WWOX, TP53, DPH1, BRCA1, mTORC2, PEG3, IGF2, SAT2, p16INK4A, p14ARF, IRF3/7, any therapeutic moiety listed in Table 3, and any combination thereof.

9. The vector of claim 1, wherein the at least one therapeutic moiety is a prodrug modifying enzyme.

10. The vector of claim 8, wherein the pathogenic gene of interest is selected from the group consisting of: an oncogene, an angiogenic gene, an immune suppressive gene, an anti-apoptotic gene, a therapy resistance gene, and any combination thereof.

11. The vector of claim 10, wherein the pathogenic gene of interest is selected from the group consisting of: MDM2, HDM2/HDM4/HDMX, BCL2, BCL-XL, BIRC5/Survivin, AKT, PLK1, E2F1, CMYC, NFKappaB, CCND1-3, KRAS, FOS, JUN, JAK, STAT, PKA, CREB, TGFB, Beta-Catenin, IL10, PD1, PDL1, MET, AXL, SRC, RAS, PIK3, PGP2, GST, MRP1, BCRP/ABCG2, any gene listed in Table 1, and any combination thereof.

12. The vector of claim 11, wherein the genome editing endonuclease is a CRISPR-associated endonuclease.

13. The vector of claim 12, wherein the CRISPR-associated endonuclease is selected from the group consisting of: Cas9, Cas12a, Cas12b, Cas12e, Cas13a, Cas13b, Cas14, Cas-theta, CasX, CasY, any CRISPR-associated endonuclease listed in Table 2, and any combination thereof.

14. The vector of claim 13, wherein the one or more gRNA sequences is selected from any gRNA comprises a nucleic acid sequence corresponding to SEQ ID NO: 81 described in Table 1.

15. The vector of claim 14, wherein the genome editing endonuclease is Cas9.

16. The vector of claim 1, wherein the vector is a lipid nanoparticle.

17. The vector of claim 1, wherein the vector is a viral vector.

18. The vector of claim 17, wherein the viral vector is selected from the group consisting of: an adenovirus, an adeno-associated virus, a lentivirus, a herpes virus, and any derivative thereof.

19. A method of treating a multigenic disease in a subject in need thereof, the method comprising administering an effective amount of a vector composition that (i) disrupts a pathogenic gene and (ii) expresses at least one therapeutic gene or therapeutic moiety.

20.-26. (canceled)

27. A composition comprising:
(a) a one donor therapeutic TP53 nucleic acid sequence;
(b) a genome editing endonuclease or a nucleic acid sequence encoding a genome editing endonuclease; and
(c) one or more gRNA sequences for guiding the genome editing endonuclease to cleave an MDM2 gene,
the composition providing for (i) disruption of the MDM2 gene and (ii) expression of a therapeutic gene or a therapeutic moiety from the donor therapeutic TP53 nucleic acid sequence.
